(19) United States
(12) Patent Application Publication
(10) Pub. No.: US 2023/0263831 A1
(43) Pub. Date: Aug. 24, 2023

(54) METHODS FOR ENHANCING ANTI-TUMOR ACTIVITY OF EXHAUSTED T CELLS

(71) Applicant: FRED HUTCHINSON CANCER CENTER, Seattle, WA (US)

(72) Inventors: Geoffrey Hill, Seattle, WA (US); Simone Minnie, Seattle, WA (US)

(73) Assignee: FRED HUTCHINSON CANCER CENTER, Seattle, WA (US)

(21) Appl. No.: 18/173,485

(22) Filed: Feb. 23, 2023

Related U.S. Application Data
(60) Provisional application No. 63/313,153, filed on Feb. 23, 2022.

(51) Int. Cl. A61K 35/17; A61P 35/00; A61K 38/20

(52) U.S. Cl. CPC A61K 35/17; A61P 35/00; A61K 38/20

(57) ABSTRACT

Methods for treating malignancies including multiple myeloma (MM), methods for expanding immune cells, methods for characterizing and enhancing anti-tumor functions of immune cells, and methods for characterizing immune cell responses to agonist immunotherapies including decoy-resistant IL-18 (DR-18) therapies. The methods include administering a composition to enrich for a precursor exhausted population with stem-like properties and the ability for self-renewal (T_{PEX} cells), which is crucial for sustaining an immune response to chronic infection and tumors T_{PEX} cells and/or TOX⁺T_{EFF} cells

### CROSS-REFERENCE TO RELATED APPLICATION

This application claims the benefit of U.S. Provisional Application No. 63/313,153, filed Feb. 23, 2022, the disclosure of which is hereby expressly incorporated by reference in its entirety.

### STATEMENT OF GOVERNMENT LICENSE RIGHTS

This invention was made with government support under CA244291, awarded by the National Institutes of Health. The government has certain rights in the invention.

### BACKGROUND

Previous progress toward understanding CD8 T-cell responses to infection and cancer have led to the development of the concept of “T-cell exhaustion”, a perceived state of a T-cell in which the T-cell is dysfunctional, i.e., one or more functions of the T-cell (e.g., cytokine production, killing function) appear to be deteriorated. Importantly, the description and understanding of CD8 T-cell exhaustion is constantly evolving due to the discovery of novel transcription factors associated with exhausted phenotypes (T_{EX}). Most recently, the gain of expression of TOX and NR4A family transcription factors and the loss of TCF-1 expression have been associated with a T_{EX} phenotype in mice and humans. Maintenance of TCF-1 expression marks a precursor exhausted population (T_{PEX}) with stem-like properties and the ability for self-renewal, which is crucial for sustaining an immune response to chronic infection and tumors.

Despite debate surrounding the overall translatability of exhaustion signatures driven by chronic viral infection to the tumor microenvironment (TME), there has been a consensus that CD8 T-cells that express multiple inhibitory receptors are not functional after ex vivo restimulation in either setting. However, the increasing complexity of transcriptional networks (e.g. BATF, IRF4, NFAT) that have been shown to drive both a cytotoxic effector or exhaustion phenotype suggests that broad use of the terminology ‘dysfunctional’ to describe CD8 T-cells with an exhausted surface marker or transcriptional profile may not capture functional nuances.

Accordingly, an improved understanding of CD8 T-cells and T-cell exhaustion is needed for the continued development and validation of immunotherapies targeting cancer and other conditions. The present disclosure address this and other long-felt and unmet needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In a first aspect, the disclosure provides a method for treating a malignancy in a subject, the method comprising contacting a composition to enrich for a precursor exhausted population (T_{PEX}) with stem-like properties and the ability for self-renewal, which is crucial for sustaining an immune response to chronic infection and tumors. The method enriches for TOX⁺T_{EFF} cells within a plurality of T cells to enhance anti-tumor activity of the plurality of T cells to treat the malignancy.

In a second aspect, the disclosure provides a method for determining whether enhancement of an anti-tumor activity of a plurality of T cells associated with a tumor microenvironment (TME) of a malignancy occurs in a subject, the method comprising: administering a composition to the subject to enrich for T_{PEX} cells and/or TOX⁺T_{EFF} cells in the plurality of T cells; and determining, with an assay, whether the anti-tumor activity is enhanced as a result of administering the composition. The method is advantageously performed in vivo and does not utilize ex vivo restimulation, which allows for a more accurate determination of the anti-tumor activity in the absence of potential experimental or procedural artifacts due to ex vivo restimulation.

In embodiments, the composition comprises an agonist immunotherapy or a decoy-resistant IL-18 (DR-18) immunotherapy. In embodiments, the DR-18 treatment is administered according to an experimental regimen or according to a therapeutic regimen. In embodiments, the DR-18 treatment is administered with one or more therapeutically effective doses, optionally in combination with one or more other treatments. In embodiments, the contacting the composition expands IFNγ⁺TOX⁺T_{EFF} cells and promotes tumor-specific immunity. In embodiments, the contacting the composition expands Maf-expressing TOX⁺T_{EFF} cells with a tumor-specific gene signature. In embodiments, the TOX⁺T_{EFF} cells express Basic leucine zipper transcription factor, ATF-like (BATF). In embodiments, the composition is administered to the subject in vivo and enrichment for T_{PEX} cells and/or TOX⁺T_{EFF} cells in the plurality of T cells occurs in vivo.

In embodiments, the administering the composition to the subject increases survival of the subject relative to not administering the composition. In embodiments, the administering the composition occurs prior to a state of high tumor burden of the malignancy.

In at least some embodiments, the method further comprises administering a restimulation composition to restimulate the plurality of T cells. In embodiments, the restimulation composition comprises phorbol 12-myristate 13-acetate (PMA) and ionomycin.

In embodiments, the subject is a cytokine reporter animal model. In other embodiments, the subject is a different animal model, a clinical trial subject, or a clinical patient, e.g., a human clinical patient. In at least some embodiments utilizing the cytokine reporter animal model, the assay measures at least one reporter protein of the cytokine reporter animal model for determining whether enhancement of the anti-tumor activity occurs as a result of administering the composition to the subject.

### DESCRIPTION OF THE DRAWINGS

The patent or application file contains at least one drawing executed in color. Copies of this patent or patent application publication with color drawing(s) will be provided by the Office upon request and payment of the necessary fee.

The foregoing aspects and many of the attendant advantages of this disclosure will become more readily appreciated as the same become better understood by reference to the following detailed description, when taken in conjunction with the accompanying drawings, wherein:

FIG. 1A shows UMAP embedding of RNA data from CD8 T-cells isolated from BM of mice with relapsed myeloma (n=4 mice pooled). Cells are colored by cluster with RNA velocity vectors depicting differentiation trajectory.

FIG. 1B shows CD8 T-cells colored by subsets based on cell surface markers expression (CITE-seq) of TIM-3 and PD-1.

FIG. 1C shows a dot plot depicting gene expression in TIM-3 and PD-1 subsets.

FIG. 1D shows representative flow cytometry plots of TOX and c-Maf expression in TIM-3 and PD-1 subsets from BM. Expression of TOX (n=12; Kruskal-Wallis test with Dunn's test), c-Maf (n=6), granzyme B (n=4), and perforin (n=6).

FIG. 1E shows IFNγ and IL-10 reporter protein expression in subsets with representative plots (n=19; Two-Way ANOVA with Sidak's test).

FIG. 1F shows MFI of IFNγ in IFNγ⁺ cells in each subset (n=12). (FIGS. 1D, 1E, and 1F) Data is mean±SEM. Each symbol represents an individual mouse. One-Way ANOVA with Tukey's test unless otherwise stated. *p<0.05, **p<0.01, ***p<0.001. In summary, FIGS. 1A-1F show that TIM-3⁺TOX⁺CD8 T-cells produce cytolytic molecules and IFNγ in vivo.

FIG. 2A shows WNN embedding of concurrent ATAC and RNA data of CD8 T-cells isolated from BM of mice that were never injected with tumor (MM-free; n=2 pooled), had controlled tumor (MM-controlled; n=3 pooled) or had progressive disease (MM-relapsed; n=6 pooled) with cells colored by cluster (top) or by cohort (bottom).

FIG. 2B shows pseudotime analysis from naïve (TN) to Tox⁺ clusters.

FIG. 2C shows gene expression (top) and chromatin accessibility (bottom) of key functional proteins over pseudotime.

FIG. 2D shows experiment wide regulation score transcription factors (TFs). TFs with positive scores have inferred activating capacity and negative scores have inferred suppressive activity.

FIG. 2E shows inferred activating (top right) and repressing (top left) transcription factors of Havcr, Pdcd1, IFNγ, and Prf1.

FIG. 2F shows gene expression of Tbx21, Eomes, Batf and Tox.

FIG. 2G shows single cell accessibility of Batf binding domain measured by ChromVAR. In summary, FIGS. 2A-2G show TOX⁺T_{EFF} capacity to produce cytokines and cytotoxic molecules is positively regulated by Batf.

FIG. 3A shows representative flow cytometry plots and frequency of CD8 T-cells expressing IFNγ reporter protein in BM after treatment with decoy-resistant IL-18 (DR-18; n=10) or PBS (MM-controlled n=6; MM-relapsed n=4).

FIG. 3B shows frequency of precursor exhausted (T_{PEX}; PD-1⁺Ly108^{High}) cells.

FIG. 3C shows frequency of TIM-3⁺TOX⁺ cells.

FIG. 3D shows MFI of IFNγ reporter protein in TIM-3 and PD-1 subsets.

FIG. 3E shows a representative multiplex immunohistochemistry image of healthy BM (PBS=i; DR-18=iii) and myeloma lesion (PBS=ii; DR-18=iv) immune cell infiltration after treatment.

FIG. 3F shows M-band and overall survival of PBS (n=10), IL-18 (n=13), and DR-18 (n=13) treated mice.

FIG. 3G shows survival of DR-18 treated mice after rechallenge (arrows indicate tumor injection) with the original (Vk12653) or a different (Vk12598) myeloma clone compared to respective naïve controls for each rechallenge. DR-18 treated mice were long-term controllers from FIG. 3F. For FIGS. 3A-3G, Data are mean±SEM. Each symbol represents an individual mouse. One-Way ANOVA with Tukey's test. Mixed effects longitudinal modelling with shaded confidence intervals for M-band and Log-rank test for survival *p<0.05, **p<0.01, ***p<0.001. In summary, FIGS. 3A-3G show Decoy-resistant IL-18 expanded IFNγ⁺TOX⁺T_{EFF} cells and promotes myeloma-specific immunity.

FIG. 4A shows UMAP embedding of RNA data from CD8 T-cells from BM of PBS treated mice with relapsed (n=3; PBS rel.) or controlled (n=2; PBS con.) myeloma and DR-18 treated mice that were resistant (n=3; DR-18 rel.) or responsive (n=3; DR-18 con). Cells are colored by cluster with RNA velocity vectors depicting differentiation trajectory.

FIG. 4B shows quantification of clusters across groups.

FIG. 4C shows embedding of TOX⁺T_{EFF}_3 cluster and bar graph showing cluster frequency and violin plot showing Maf gene expression across groups.

FIG. 4D shows a dot plot showing Fasl, Il10, IFNγ, and Prf1 gene expression in the TOX⁺T_{EFF}_3 cluster in DR-18 con. vs. DR-18 rel. mice.

FIG. 4E shows gene expression of neoantigen CD8 T-cell signature from human cancers by Lowery et al., Science, 2022, PMID: 35113651 (Human_CD8_NeoTCR) and violin plot of gene score across each cluster. In summary, FIGS. 4A-4E show DR-18 expands Maf-expressing TOX⁺T_{EFF} cells with a tumor-specific gene signature.

FIG. 5A shows a representative flow cytometry plot of T-cell subsets sorted for RNA sequencing with UMAP embedding of RNA data colored by sorted T-cell subsets.

FIG. 5B shows heatmaps of average gene expression across identified clusters.

FIG. 5C shows feature plots showing gene expression of Tox, Maf Tcf7, Batf, Gzmb, Prf1 (perforin), Gzma, and IFNγ.

FIG. 5D shows pseudo single cell projection of tumor-specific (SV40; exhausted phenotype) and bystander (OT1; effector phenotype) bulk RNA expression data from Scott et al., Nature, 2019, PMC7698992 (Schietinger_T_{EX}).

FIG. 5E shows Log sum gene expression of Tox+ exhausted gene set from Khan et al., Nature, 2019, PMC6713202 (Wherry_T_{EX}). In summary, FIGS. 5A-5E show myeloma generates both conserved and unique T_{EX} signatures in the bone marrow.

FIG. 6A shows a representative flow cytometry plot and frequency of IFNγ⁺IL-10⁻ and IFNγ⁺IL-10⁺CD8 T-cells in the BM of mice never injected with tumor (MM-free; n=8) and mice with controlled (MM-controlled; n=10) or progressive myeloma (MM-relapsed; n=19) at 6-7 weeks post-transplant. One-Way ANOVA with Tukey's test.

FIG. 6B shows a representative flow cytometry plot and frequency of TIM-3⁻PD-1⁺ and TIM-3⁺PD-1⁺CD8 T-cells in the BM of MM-controlled (n=9) and MM-relapsed (n=18) mice. Two-Way ANOVA with Tukey's test.

FIG. 6C shows frequency of IFNγ⁺IL-10⁻ and IFNγ⁺IL-10⁺ cells within subsets based on TIM-3 and PD-1 expression in MM-controlled mice (n=8). Two-Way ANOVA with Tukey's test.

FIG. 6D shows frequency of TIM-3⁻PD-1⁺ and TIM-3⁺PD-1⁺CD8 T-cells in bone marrow aspirates at 3 weeks post-transplant in mice that went on to develop relapsed disease. Mann-Whitney test.

FIG. 6E shows experimental design.

FIG. 6F shows frequency of TIM-3⁻PD-1⁺ and TIM-3⁺PD-1⁺CD8 T-cells in naïve HULK mice (n=3) and HULK mice with moribund myeloma (MM-bearing; n=5). Two-Way ANOVA with Tukey's test.

FIG. 6G shows frequency of IFNγ⁺IL-10⁻ and IFNγ⁺IL-10⁺ cells within subsets based on TIM-3 and PD-1 expression in MM-bearing mice (n=5). Two-Way ANOVA with Tukey's test. For FIGS. 6A-6G, All data is mean±SEM. Each symbol represents an individual mouse. *p<0.05, **p<0.01, ***p<0.001. In summary, FIGS. 6A-6G show that CD8 T-cells from an active tumor microenvironment produce the most IFNγ in vivo.

FIG. 7A shows an experimental design.

FIG. 7B shows single cell gene accessibility and expression of Il10 (left) and IFNγ (right).

FIG. 7C shows ATAC-seq coverage plots at Il10 and IFNγ loci. Peaks uniquely gained or with increased accessibility in exhausted clusters are highlighted in blue. In summary, FIGS. 7A-7C show mice with relapsed myeloma have increased chromatin accessibility in IFNγ and Il10 genes.

FIG. 8 shows heatmaps demonstrating changes in chromatin accessibility (left) and gene expression (right) over pseudotime from naïve/stem-like states to an exhausted phenotype. Arrows indicate key genes. In summary, TOX⁺ T-cells have an epigenetic and transcriptional signature associated with exhaustion in relapsed myeloma.

FIG. 9A shows UMAP embedding of CD8 T-cells from Zheng, Qin, Si et al. Science, 2021 generated using publicly available online tool.

FIG. 9B shows a heatmap of gene expression, including key genes from our mouse dataset, in human TILs.

FIG. 9C shows Feature plots showing gene expression on a single cell level for TBX21, EOMES, BATF, TOX, HAVCR2 (TIM-3), GZMB, PRF1 (perforin), and IFNγ. In summary, FIGS. 9A-9C show Batf is co-expressed with Tox and functional molecules in tumor-infiltrating lymphocytes in humans.

FIG. 10A shows an experimental design.

FIG. 10B shows FlowSOM heatmaps showing MFI of markers across populations (left) and average relative frequencies of each population across treatment groups (right) at 6 weeks post-transplant. PBS-treated mice with controlled disease=PBS (con.); n=6; green. PBS-treated mice with relapsed myeloma=PBS (rel.); n=4; purple. All DR-18 treated mice had controlled myeloma=DR-18(con.); n=10; orange.

FIG. 10C shows frequency of IFNγ⁺CD8 T-cells within CD45⁺ cells (left) and MFI of IFNγ in these cells (right) in bone marrow aspirates (BMA) at 3 weeks post-transplant.

FIG. 10D shows frequency of IFNγ⁺ cells within CD8 T-cells measured by intracellular cytokine staining after PMA/ionomycin restimulation (Restim) and by reporter protein in unstimulated cells at 5 weeks post-transplant.

FIG. 10E shows frequency of IFNγ⁺ cells within CD8 T-cell subsets based on TIM-3 and PD-1 expression. One-way ANOVA with Tukey's test.

FIG. 10F shows IFNγ reporter MFI in TIM-3⁺PD-1⁺ cells.

FIG. 10G shows frequency of perforin⁺ (Pfp), granzyme A⁺ (GrzA), granzyme B⁺ (GrzB) and GrzA⁺GrzB⁺ cells measured by intracellular staining without restimulation in CD8 T-cell subsets based on TIM-3 and PD-1 expression. *p<0.05, **p<0.01, *** p<0.001. In summary, FIGS. 10C, 10D, 10E, 10F, and 10G show CD8 T-cell phenotype summary data from BM of mice treated with PBS (n=8) or DR-18 (n=8). Data is mean 15±SEM. Mann-Whitney test unless otherwise stated. FIGS. 10A-10G show DR-18 expands and enhances function of IFNγ⁺TOX⁺T_{EFF} cells in the bone marrow TME.

FIG. 11 shows heatmaps of gene expression across T-cell clusters identified in BM of PBS-treated mice with controlled (n=2) or relapsed myeloma (n=2) and in mice sensitive (n=3) or resistant (n=3) to DR-18 treatment. In summary, DR-18 resistance is associated with loss of a TOX⁺T_{EFF} subset expressing Maf.

FIG. 12 shows embedding of RNA sequencing data from CD8 T-cells from BM of mice with relapsed myeloma overlaid with gene expression of neoantigen CD8 T-cell signature from human cancers by Lowery et al., Science, 2022, PMID: 35113651 (Human_CD8_NeoTCR). Violin plot of gene score within each cluster identified in murine dataset. In summary, CD8 T-cells from mice with relapsed myeloma express gene signature associated with tumor-specific T-cells in human cancer.

### DETAILED DESCRIPTION

The disclosure provides methods for treating malignancies, including but not necessarily limited to hematological malignancies, such as multiple myeloma. The disclosure also provides methods for analyzing or characterizing immune cells in vivo, in the context of an agonist immunotherapy for treatment of cancer. In embodiments, the agonist immunotherapy comprises an established or approved immunotherapy, or alternatively, an experimental immunotherapy. In embodiments, the agonist immunotherapy is a DR-18 treatment and includes administering a composition that comprises DR-18 to a subject.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

The phrase “pharmaceutically acceptable carrier” is art recognized and includes a pharmaceutically acceptable material, composition, or vehicle, suitable for administering compounds used in the methods described herein to subjects, e.g., mammal subjects or human subjects. The methods herein can include administration of one or more agents that are formulated with one or more pharmaceutically acceptable carriers to a subject.

The language “therapeutically effective amount” or a “therapeutically effective dose” of a compound is the amount necessary to or sufficient to provide a detectable improvement of at least one cause of and/or symptom associated with or caused by the condition, disease, or disorder being treated. The therapeutically effective amount can be administered as a single dose or in multiple doses over time. Two or more compounds can be used together to provide a “therapeutically effective amount” to provide a detectable improvement wherein the same amount of either compound alone would be insufficient to provide a therapeutically effective amount. “Therapeutically effective amount,” as used herein refers to an amount of an agent which is effective, upon single or multiple dose administration to the cell or subject, decreasing at least one sign or symptom of the disease or disorder, or prolonging the survivability of the patient with such a disease or disorder beyond that expected in the absence of such treatment.

An agent can be administered to a subject, either alone or in combination with one or more therapeutic agents, as a pharmaceutical composition in mixture with conventional excipient, e.g., pharmaceutically acceptable carrier. The agent can be administered using any suitable method or mode of administration, including but not limited to parenteral administration, injection, intravascular injection, intravenous injection, infusion (e.g., using a pump), and the like. A composition can be administered according to any suitable regimen, for example, once a day, once a week, every two weeks, once a month, or more or less frequently, depending on the specific needs of the subject to be treated. The specific pharmacokinetic and pharmacodynamic properties of the composition to be administered can affect dosing. Such administration can be used as a chronic or acute therapy. The amount of active ingredient that can be combined with the carrier materials to produce a single dosage form can vary depending upon the host treated and the particular mode of administration.

Cells and/or subjects can be treated and/or contacted with one or more standard cancer therapeutic treatments, including but not limited to surgery, chemotherapy, radiotherapy, gene therapy, immune therapy, anti-angiogenic therapy, hormonal therapy, tissue transplant, blood transplant, bone marrow transplant, and/or another therapy or treatment, for example, as may be prescribed by a health care provider.

A “decoy resistant” IL-18 (DR-18) agent is a variant, mutant, or mimic of interleukin-18 (IL-18) that binds to IL-18 receptor (IL-18R), thereby inducing/enhancing/stimulating IL-18 signaling activity, but exhibits little to no binding to the inhibitory IL-18 binding protein (IL-18BP). DR-18 agents are therefore IL-18R agonists that are resistant to inhibition by IL-18BP. Examples of DR-18 agents, and of methods of making and using DR-18 agents, are described in U.S. Pat. App. Pub. No. 2019/0070262 A1 and U.S. Pat. App. Pub. No. 2021/0015891 A1, both of which are incorporated herein by reference in their entirety. A decoy-resistant IL-18 (DR-18) treatment includes any treatment that includes administration of one or more DR-18 agents to a subject.

### Methods for Treating Malignancies

In a general sense, the disclosure provides methods for treating malignancies in subjects. The methods comprise contacting a composition to a plurality of T cells to enrich for a precursor exhausted population (T_{PEX}) of T cells having stem-like properties and the ability for self-renewal, and/or TOX⁺T_{EFF} cells. The contacted immune cells activate and sustain an immune response against chronic infection and, in the case of a malignancy, tumors of the malignancy. The method thereby enhances anti-tumor activity of the plurality of T cells to treat the malignancy.

In embodiments, the method comprises contacting the T cells with a composition that comprises a therapeutically effective amount of an agonist immunotherapy or a decoy-resistant IL-18 (DR-18) immunotherapy. In embodiments, the DR-18 treatment is administered according to an experimental regimen or, alternatively, according to a therapeutic regimen. In embodiments, the DR-18 treatment is administered with one or more therapeutically effective doses, optionally in combination with one or more other treatments. Examples of other treatments include other anti-malignancy treatments, anti-cancer treatments, and the like. Accordingly, in embodiments, the DR-18 treatment is administered alone or in combination with one or more standard cancer therapeutic treatments, including but not limited to surgery, chemotherapy, radiotherapy, gene therapy, immune therapy, anti-angiogenic therapy, hormonal therapy, tissue transplant, blood transplant, bone marrow transplant, and/or another therapy or treatment, for example, as may be prescribed by a health care provider.

While a DR-18 treatment, a cancer treatment, both, and/or one or more other agonist immunotherapies promote immune cell activation and increase anti-tumor immunity, in at least some instances, a beneficial agonist immunotherapy causes and/or is associated with any of the following characteristics, in whole or in part or in any combination thereof: (1) expansion of IFNγ⁺TOX⁺T_{EFF} cells; (2) promotion of tumor-specific immunity; (3) expansion of Maf-expressing TOX⁺T_{EFF} cells with a tumor-specific gene signature; and (4) expansion of TOX⁺T_{EFF} cells that express Basic leucine zipper transcription factor, ATF-like (BATF).

In embodiments, the methods include and/or are combined with one or more other established or experimental treatments. For example, in embodiments, a method further comprises administering a tissue, a stem cell, or a donor lymphocyte infusion (DLI) to the subject. A DLI is a blood cell infusion in which CD3⁺ lymphocytes from a donor are infused, after a tissue (e.g., bone marrow) transplant, to augment an anti-tumor immune response or ensure that the donor cells remain engrafted. Generally, the donated white blood cells contain cells of the immune system that recognize and destroy cancer cells.

In embodiments, the composition is administered to the subject in vivo and enrichment for T_{PEX} cells and/or TOX⁺T_{EFF} cells in the plurality of T cells occurs in vivo. Accordingly, it is unexpectedly and surprisingly disclosed herein that populations of T cells previously thought to be exhausted are able to be enriched for T cells having tumor-specific immunity for treatment or management of the malignancy. As such, in at least some embodiments, the administering the composition to the subject increases survival of the subject relative to not administering the composition, as determined with one or more control groups. In at least some embodiments, the administering the composition occurs prior to a state of high tumor burden of the malignancy, and in this manner, tumor-specific immunity is available at the onset of tumor growth and the tumor is more effectively managed.

In embodiments, the method further comprises administering a restimulation composition to restimulate the plurality of T cells. In embodiments, the restimulation composition comprises phorbol 12-myristate 13-acetate (PMA) and ionomycin. PMA activates protein kinase C, and ionomycin is a calcium ionophore. In at least some instances, stimulation with these compounds bypasses the T cell membrane receptor complex and leads to activation of intracellular signaling pathways, resulting in T cell activation and production of a variety of cytokines.

In at least some embodiments, the subject is a cytokine reporter animal model, e.g., a murine model. However, in other embodiments, the subject is a different animal model, a clinical trial subject, or a clinical patient, e.g., a human clinical patient. In at least some embodiments utilizing the cytokine reporter animal model, the assay measures at least one reporter protein of the cytokine reporter animal model for determining whether enhancement of the anti-tumor activity occurs as a result of administering the composition to the subject. In embodiments, the assay is a molecular biology technique as described herein or as known in the art.

### Methods for Characterizing Immune Cells

In another general aspect, the disclosure provides methods for determining whether enhancement of an anti-tumor activity of a plurality of T cells associated with a tumor microenvironment (TME) of a malignancy occurs in a subject. The methods are useful for characterizing immune cell responses to agonist immunotherapies, such as decoy-resistant IL-18 (DR-18) therapies and provide a framework for additional development of these and other cancer therapies.

In embodiments, the method comprises administering a composition to a subject to enrich for T_{PEX} cells and/or TOX⁺T_{EFF} cells in a plurality of T cells, and determining, with an assay, whether an anti-tumor activity is enhanced as a result of administering the composition. The method is advantageously performed in vivo and does not utilize ex vivo restimulation, which allows for a more accurate determination of the anti-tumor activity in the absence of potential experimental or procedural artifacts due to ex vivo restimulation.

In embodiments, the method comprises contacting the T cells with a composition that comprises a therapeutically effective amount of an agonist immunotherapy or a decoy-resistant IL-18 (DR-18) immunotherapy. In embodiments, the DR-18 treatment is administered according to an experimental regimen or, alternatively, according to a therapeutic regimen. In embodiments, the DR-18 treatment is administered with one or more therapeutically effective doses, optionally in combination with one or more other treatments. Examples of other treatments include other anti-malignancy treatments, anti-cancer treatments, and the like. Accordingly, in embodiments, the DR-18 treatment is administered alone or in combination with one or more standard cancer therapeutic treatments, including but not limited to surgery, chemotherapy, radiotherapy, gene therapy, immune therapy, anti-angiogenic therapy, hormonal therapy, tissue transplant, blood transplant, bone marrow transplant, and/or another therapy or treatment, for example, as may be prescribed by a health care provider.

While a DR-18 treatment, a cancer treatment, both, and/or one or more other agonist immunotherapies promote immune cell activation and increase anti-tumor immunity, in at least some instances, a beneficial agonist immunotherapy causes and/or is associated with any of the following characteristics, in whole or in part or in any combination thereof: (1) expansion of IFNγ⁺TOX⁺T_{EFF} cells; (2) promotion of tumor-specific immunity; (3) expansion of Maf-expressing TOX⁺T_{EFF} cells with a tumor-specific gene signature; and (4) expansion of TOX⁺T_{EFF} cells that express Basic leucine zipper transcription factor, ATF-like (BATF).

In embodiments, the methods include and/or are combined with one or more other established or experimental treatments. For example, in embodiments, a method further comprises administering a tissue, a stem cell, or a donor lymphocyte infusion (DLI) to the subject. A DLI is a blood cell infusion in which CD3⁺ lymphocytes from a donor are infused, after a tissue (e.g., bone marrow) transplant, to augment an anti-tumor immune response or ensure that the donor cells remain engrafted. Generally, the donated white blood cells contain cells of the immune system that recognize and destroy cancer cells.

In embodiments, the composition is administered to the subject in vivo and enrichment for T_{PEX} cells and/or TOX⁺T_{EFF} cells in the plurality of T cells occurs in vivo. Accordingly, it is unexpectedly and surprisingly disclosed herein that populations of T cells previously thought to be exhausted are enriched for T cells having tumor-specific immunity for treatment or management of the malignancy. As such, in at least some embodiments, the administering the composition to the subject increases survival of the subject relative to not administering the composition, as determined with one or more control groups. In at least some embodiments, the administering the composition occurs prior to a state of high tumor burden of the malignancy, and in this manner, tumor-specific immunity is available at the onset of tumor growth and the tumor is more effectively managed.

In embodiments, the method further comprises administering a restimulation composition to restimulate the plurality of T cells. In embodiments, the restimulation composition comprises phorbol 12-myristate 13-acetate (PMA) and ionomycin. PMA activates protein kinase C, and ionomycin is a calcium ionophore. In at least some instances, stimulation with these compounds bypasses the T cell membrane receptor complex and leads to activation of intracellular signaling pathways, resulting in T cell activation and production of a variety of cytokines.

In at least some embodiments, the subject is a cytokine reporter animal model, e.g., a murine model. However, in other embodiments, the subject is a different animal model, a clinical trial subject, or a clinical patient, e.g., a human clinical patient. In at least some embodiments utilizing the cytokine reporter animal model, the assay measures at least one reporter protein of the cytokine reporter animal model for determining whether enhancement of the anti-tumor activity occurs as a result of administering the composition to the subject. In embodiments, the assay is a molecular biology technique as described herein or as known in the art.

### EXAMPLES

### Example 1. Anti-Tumor Activity in the Bone Marrow Microenvironment

One sentence summary: CD8 T-cells with an exhausted phenotype are not dysfunctional in vivo and are treated according to the present disclosure to expand CD8 T-cells and enhance anti-tumor immunity.

### Abstract

The functional state of CD8 T-cells is crucial for effective anti-tumor immunity and the development of dysfunctional tumor-specific CD8 T-cells impairs tumor control. Chronic antigen stimulation leading to T-cell exhaustion has been thought to generate dysfunctional CD8 T-cells, with the transcription factor TOX controlling the commitment to an exhausted fate. Differentiation to an exhausted state is characterized by the expression of multiple inhibitory receptors and a loss of effector function, including cytokine secretion and cytolysis. To date, immunotherapy approaches have focused on reversing this exhaustion related dysfunction. In this example, the presence of distinct TOX⁺ T-cell subsets in the bone marrow tumor microenvironment with co-expression of TIM-3, c-Maf and Batf is demonstrated. Despite an exhausted transcriptional and epigenetic profile, TOX⁺ T-cells produced granzymes, perforin, and IFNγ in vivo but lacked hallmarks of self-renewal capacity. Genes encoding proteins known to confer anti-tumor immunity were co-expressed in TOX⁺ T-cells and correlated with Batf expression in mice and humans. A decoy-resistant IL-18 expanded TOX⁺TIM-3⁺ T-cells with enhanced IFNγ production and promoted anti-tumor immunity. Thus, TOX⁺ cells expressing multiple inhibitory receptors and exhaustion-associated transcription factors include highly functional subsets that are engaged to mediate effective anti-tumor activity.

### Introduction

In this example, single cell RNA and multiome sequencing with confirmatory flow cytometry were utilized to identify trajectories of T-cell exhaustion in a murine model of myeloma. Overlapping genetic and epigenetic T_{EX} signatures previously identified in solid tumor and chronic viral infection models were observed. Next, novel mouse cytokine reporter systems, which bypass the requirement for ex vivo restimulation, were used to examine the capacity of TOX⁺CD8 T-cells to produce IFNγ, granzymes and perforin in the bone marrow (BM) TME. Interestingly, TIM-3 expression was associated with the highest expression of IFNγ, perforin and granzymes. IFNγ⁺TOX⁺ cells exhibited increased Batf expression and enhanced accessibility in the Batf motif binding domain. An analogous ‘functional’ exhaustion signature was associated with BATF expression in humans. Finally, response to agonist cytokine immunotherapy was associated with increased BM infiltration and enhanced function of TIM-3⁺TOX⁺CD8 T-cells.

### Results

### TOX⁺CD8 T-Cells Produce Cytotoxic Molecules and IFNγ In Vivo

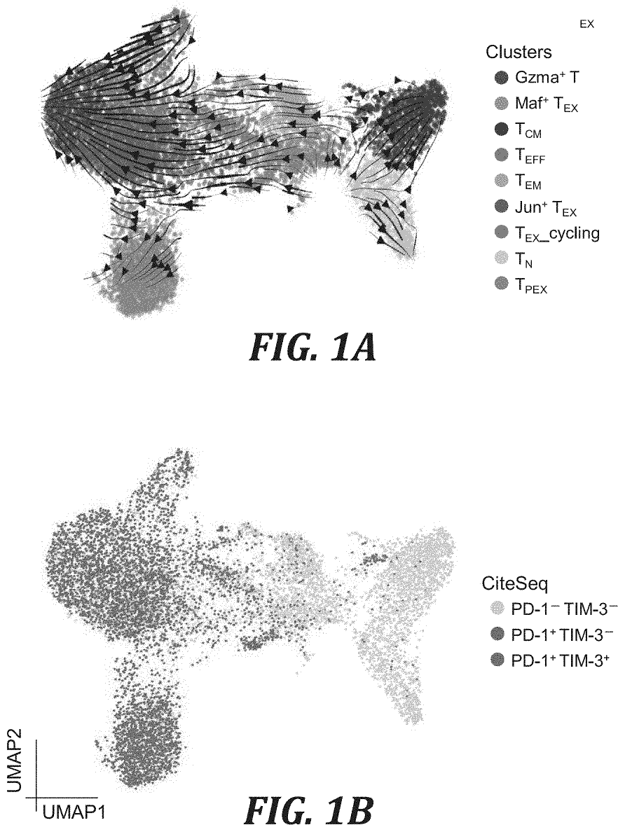

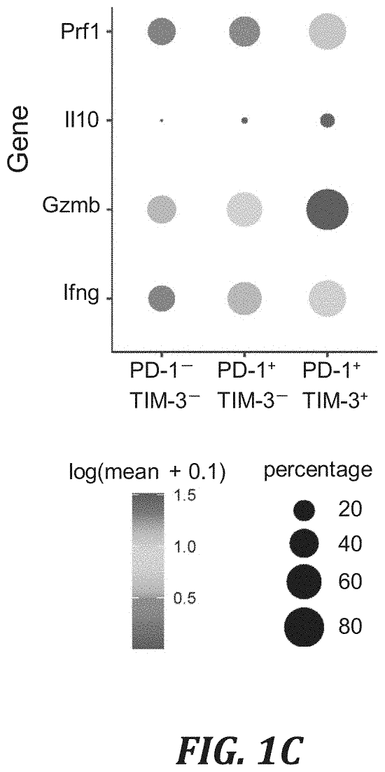

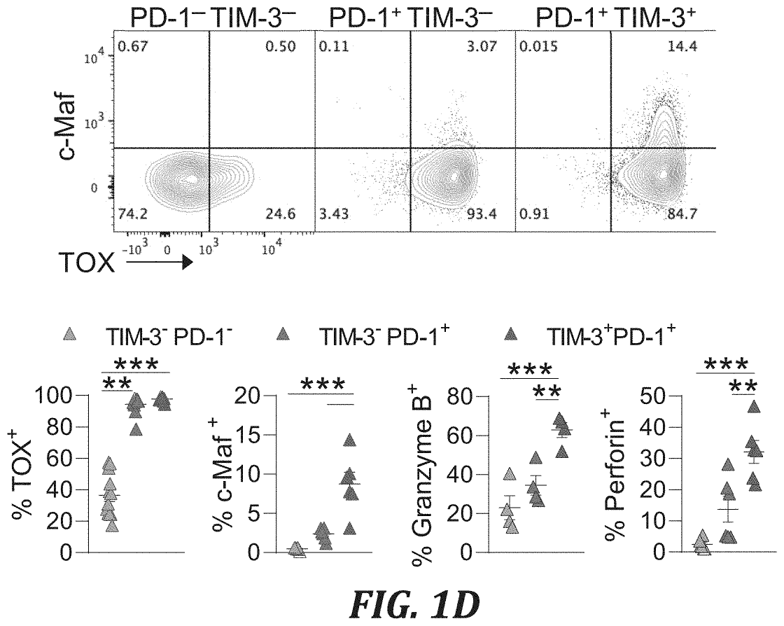

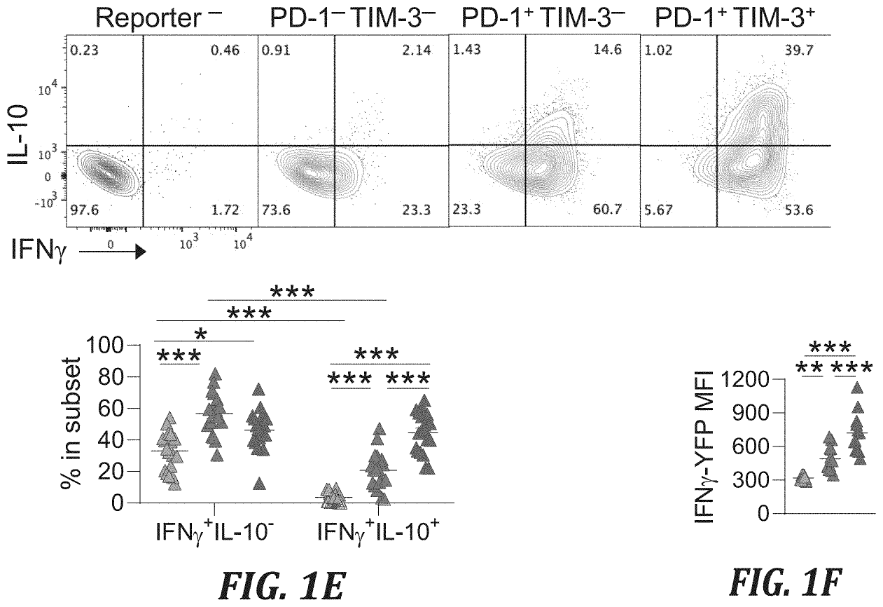

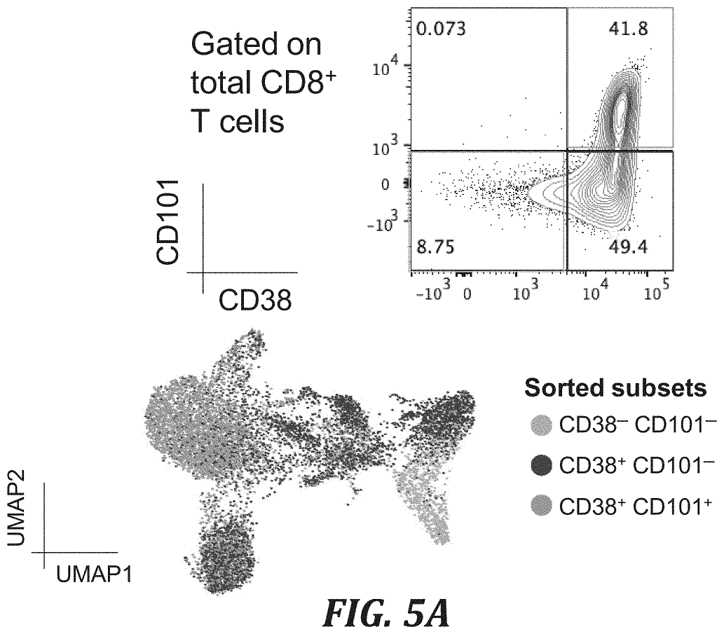

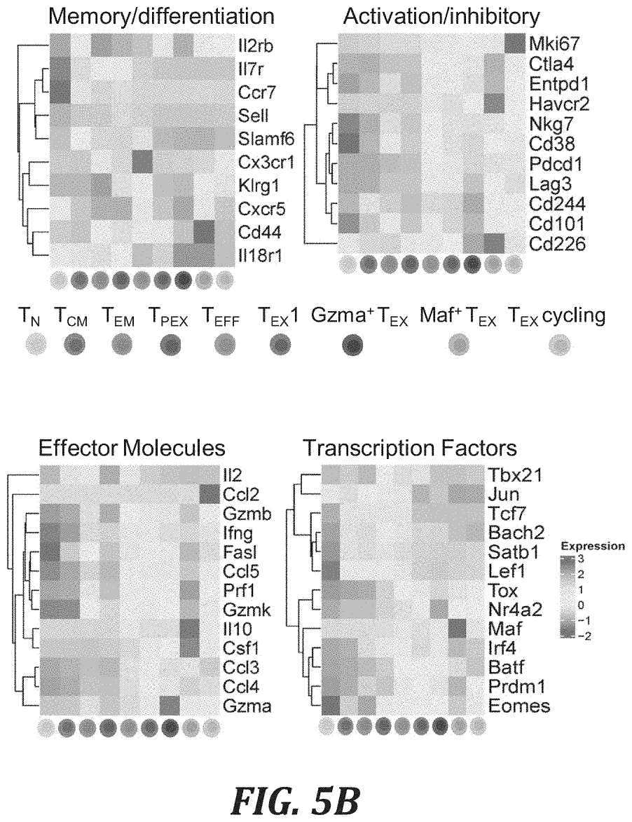

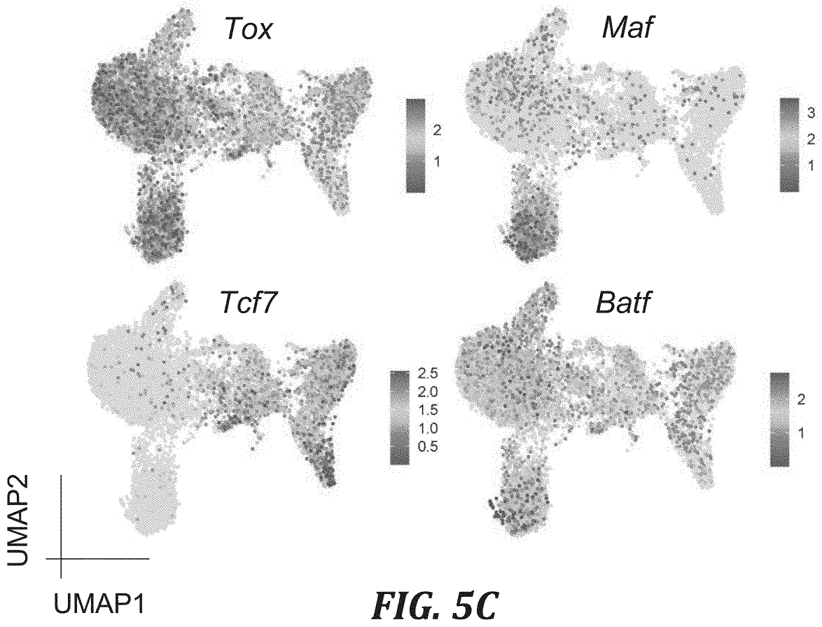

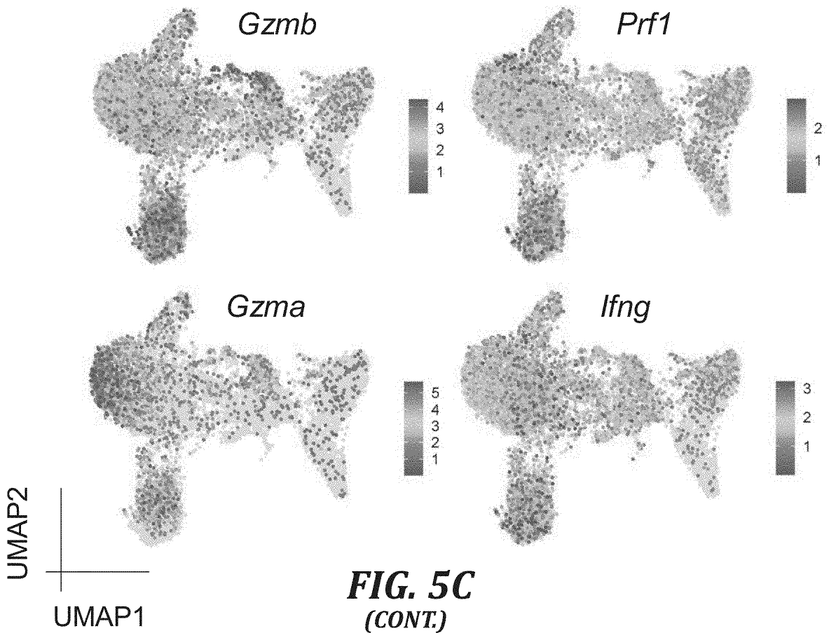

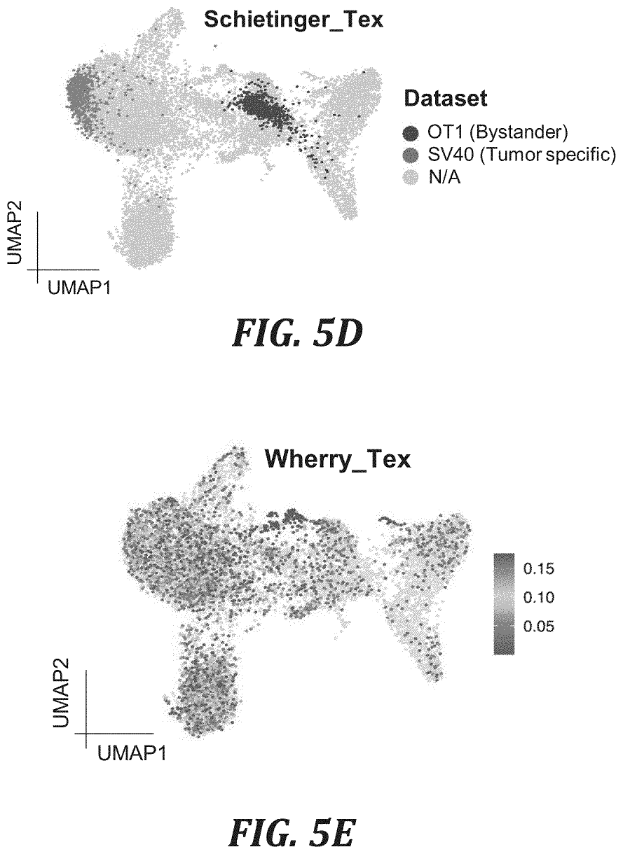

The majority of studies exploring mechanisms of T-cell exhaustion in cancer have been performed in solid tumor murine models where tumors universally relapse or regress in response to interventions. Here a tumor model is described where relapse is not universal after stem cell transplantation (SCT), which is a clinical standard of care in this disease. SCT promotes a polyclonal myeloma-specific T-cell response that is largely mediated by CD8 T-cells. Single cell RNA sequencing was utilized to capture key stages of CD8 T-cell differentiation in the BM of mice with relapsed myeloma after SCT (FIG. 1A). Briefly, CD8 T-cells were sorted into three populations based on CD38 and CD101 expression (CD38⁻CD101⁻=naïve; CD38⁺CD101⁻=activated; CD38+CD101+=exhausted) and these cells were re-pooled at an adjusted ratio (FIG. 5A). In this dataset, four populations of exhausted T-cells (T_{EX}) were characterized by expression of Tox and loss of Tcf7, which were distinguished by expression of Mki67, Maf and/or distinct cytotoxic profiles (FIGS. 5B and 5C). Interestingly, high expression of mRNA for IFNγ was observed in T_{EX} clusters, which has been described in chronic infection models (FIG. 5C). RNA velocity analysis suggests a common trajectory from a precursor exhausted (T_{PEX}) cluster towards the T_{EX} clusters in the dataset (FIG. 1A), which also resembles described pathways of T_{EX} differentiation in chronic infection and solid tumors. Furthermore, integration of CD8 T-cell exhaustion signatures into the data revealed shared gene signatures associated with a T_{EX} phenotype in solid tumor, chronic infection, and now, hematological malignancy models (FIGS. 5D and 5E). Surface expression of PD-1 and TIM-3, measured by CITEseq, was then used to subset T_{EX} cells and enhanced expression of Prf1, Gzmb and IFNγ in TIM-3⁺PD-1⁺T_{EX} cells compared to PD-1⁺TIM-3⁻ cells was unexpectedly observed (FIGS. 1B and 1C). These findings were then confirmed at the protein level with flow cytometry and it was found that all PD-1⁺CD8 T-cells expressed TOX but that cytotoxic molecules were largely found in the TIM-3⁺ subset (FIG. 1D). Expression of the transcription factor Maf was also specific to TIM-3⁺ cells by RNA and protein analyses (FIG. 1D and FIG. 5C). The TIM-3⁺T_{EX} cells identified in the model lacked Cx3cr1, Ki67, and Tbx21 (T-bet) expression and therefore are distinct from proliferating transitionary cells (FIG. 5B). Rather, the T_{EX}_cycling cluster in the dataset likely includes this transitionary subset (FIG. 1A).

Next, a triple-reporter mouse (IL-10-GFP×IFNγ-YFP×FoxP3-RFP) was used to measure cytokine production without the use of ex vivo restimulation. IFNγ reporter mice were generated with a bicistronic mRNA such that the fluorescent protein is only produced when the IFNγ mRNA is translated into protein. Expression of IFNγ-YFP in the majority of PD-1^{+l T}_{EX} cells was observed, with the greatest amount of IFNγ being produced by TIM-3⁺ cells (FIGS. 1E and 1F). Half of the TIM-3⁺T_{EX} cells also produced IL-10 (FIG. 1E). This level of cytokine production was surprising given published data, including previous results with this model, highlighting the reduced capacity of exhausted CD8 T-cells in the TME to produce IFNγ after ex vivo restimulation. These data identify a TOX⁺TIM-3⁺T_{EX} subset that produces both IFNγ and cytotoxic molecules in the TME, hereafter referred to as TOX⁺T_{EFF} cells.

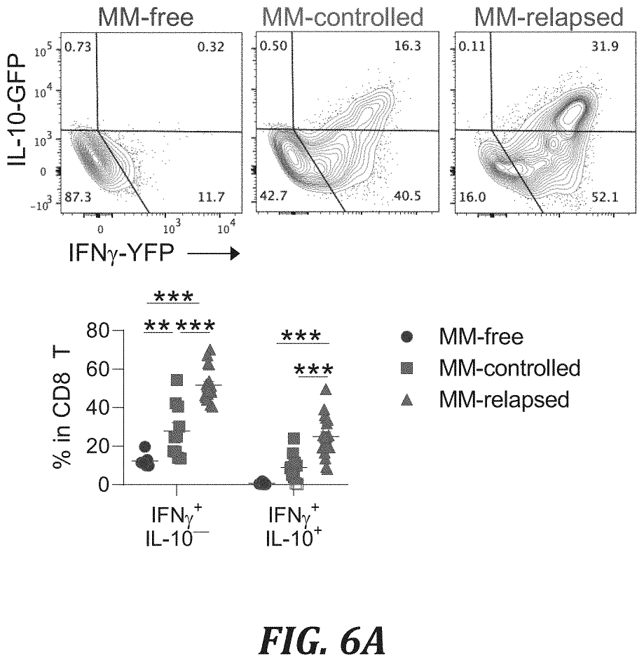

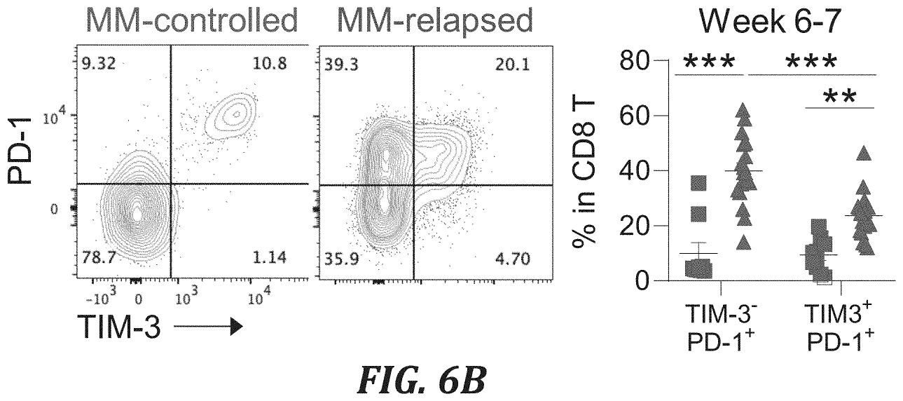

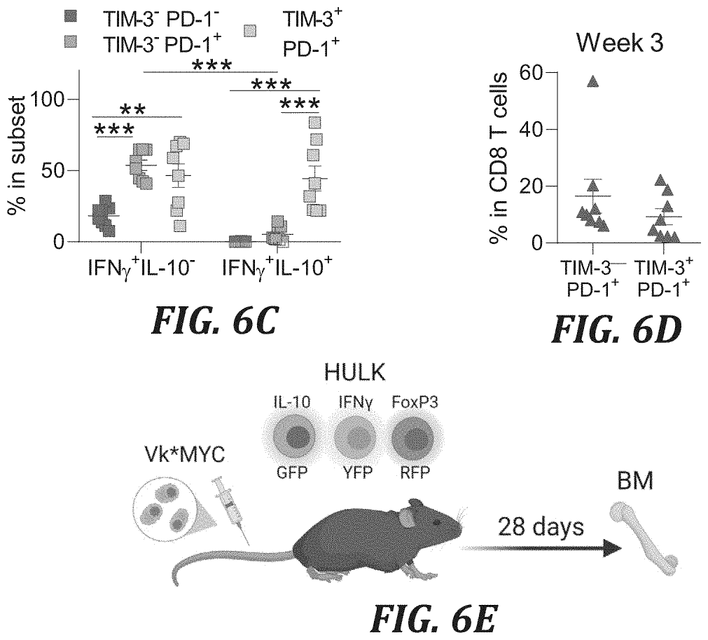

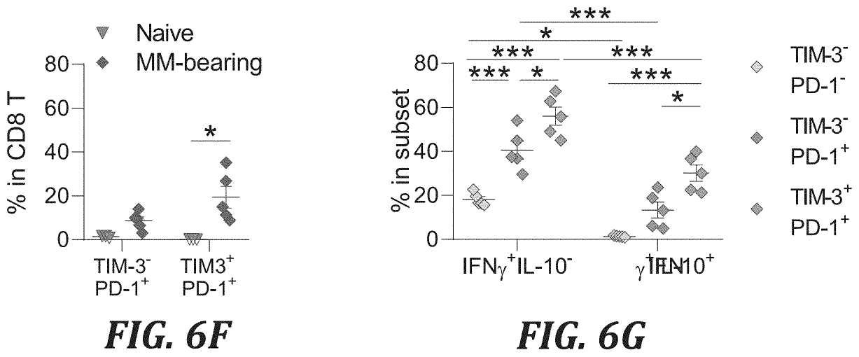

Next, the relationship between TOX⁺T_{EFF} cells and myeloma burden was delineated by phenotyping CD8 T-cells at 6-7 weeks post-transplant in mice that never received tumor (MM-free), mice that had controlled disease (MM-controlled), and mice that had progressive disease (MM-relapsed). IFNγ production was highest in MM-relapsed mice although MM-controlled mice produced more IFNγ in vivo compared to MM-free mice (FIG. 6A). Interestingly, the TOX⁺T_{EFF} cells were also found in MM-controlled mice although at a reduced frequency compared to MM-relapsed (FIG. 6B). The cytokine profile of these cells in MM-controlled mice was comparable to MM-relapsed mice (FIG. 1E, FIG. 6C). Non-terminal bone marrow aspirates (BMA) at 3 weeks post-transplant, prior to tumor progression, also revealed the presence of TIM-3⁺ cells in mice that subsequently progressed to relapse (FIG. 6D). To confirm that functional TOX⁺ T-cells were not a transplant phenomenon, CD8 T-cells from the BM of our triple-reporter mice with advanced myeloma that had not been transplanted were analyzed (FIG. 6E). A similar frequency of TOX⁺T_{EFF} cells within CD8 T-cells compared to mice that relapsed after SCT was observed, which also produced both IFNγ and IL-10 in vivo (FIGS. 6F and 6G). These data demonstrate that functional, TOX⁺T_{EFF} cells were expanded in response to tumor and that the presence of these cells in the BM preceded frank disease relapse.

### TOX⁺T_{EFF} Cells are Positively Regulated by Batf and Eomes

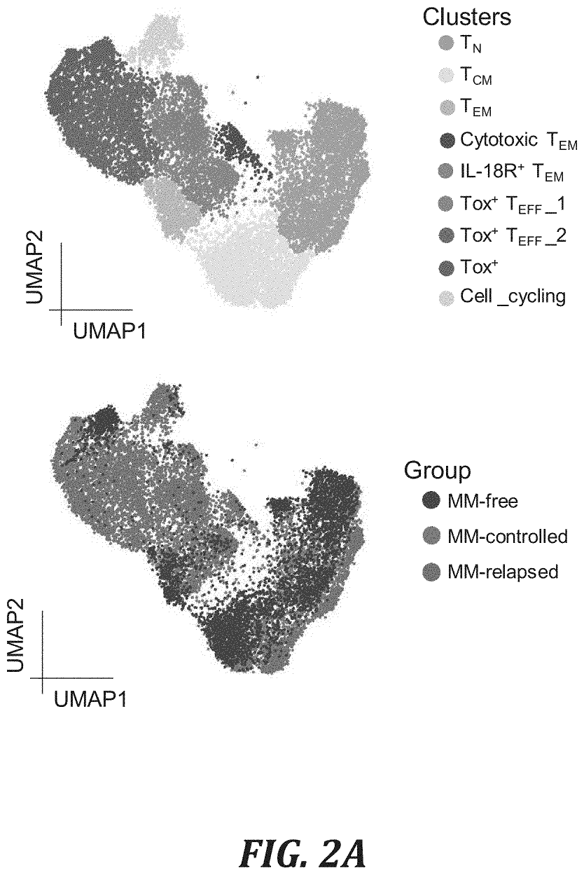

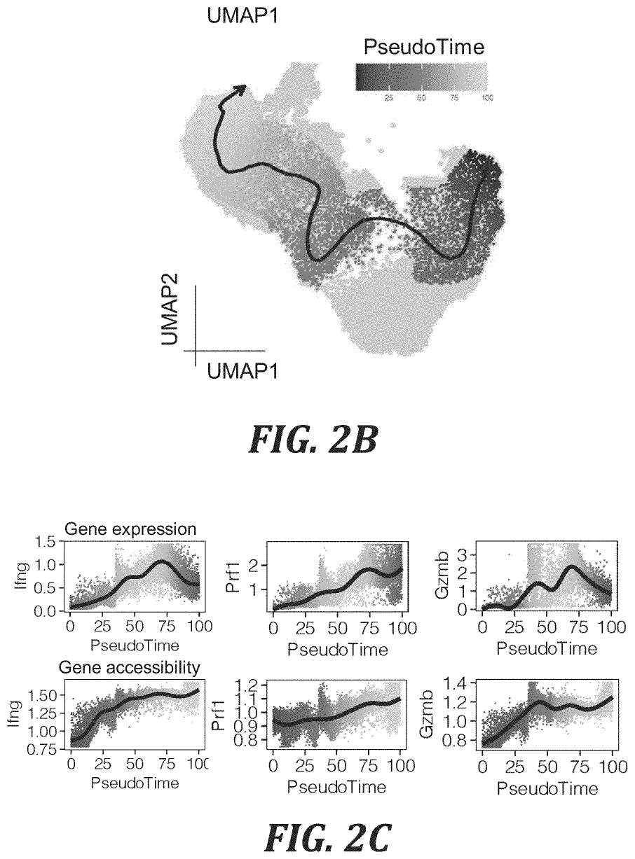

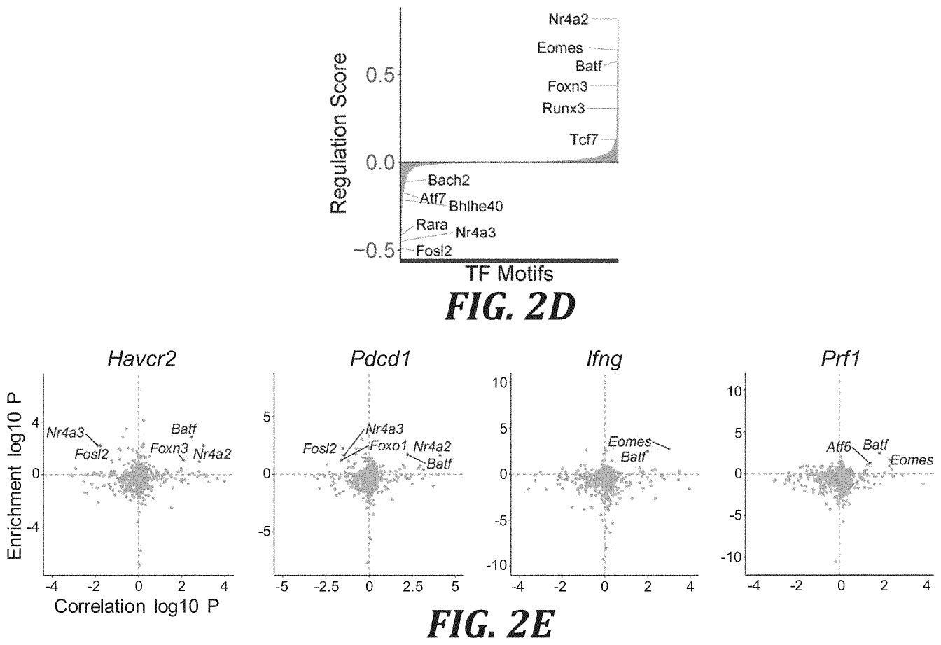

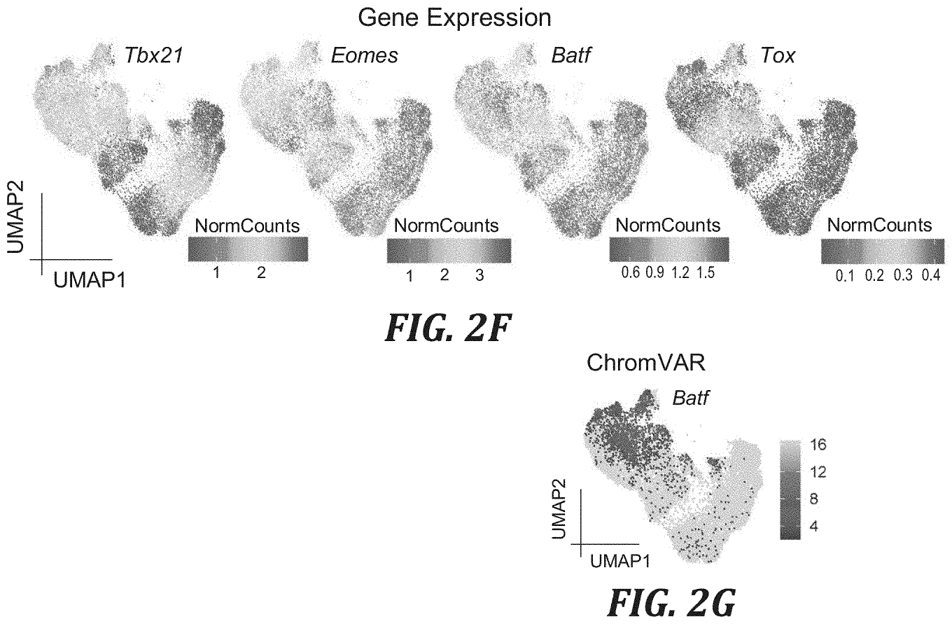

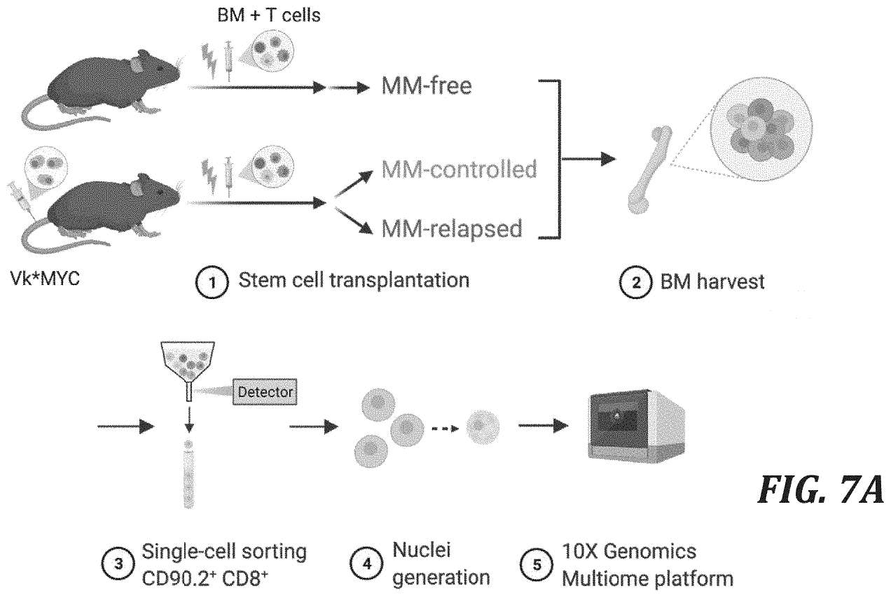

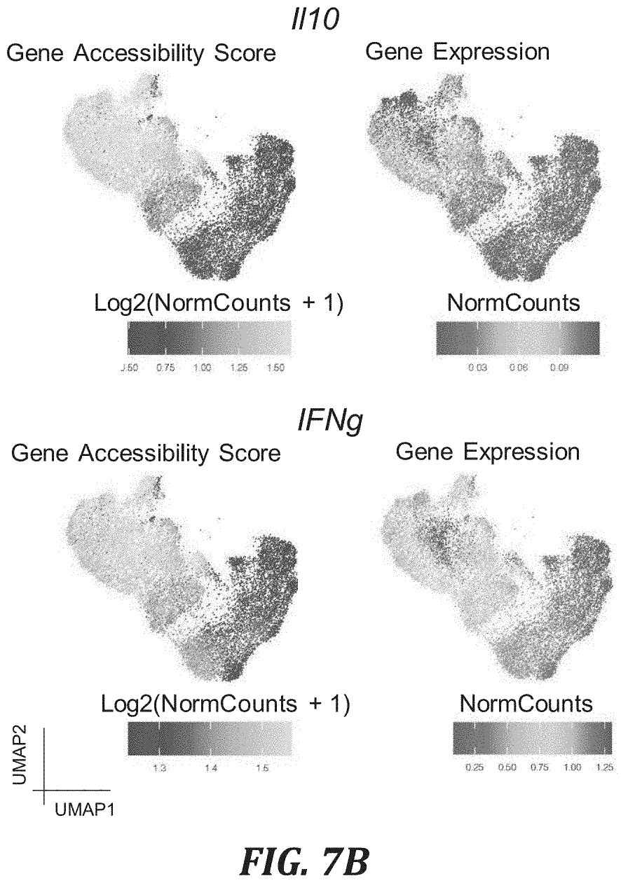

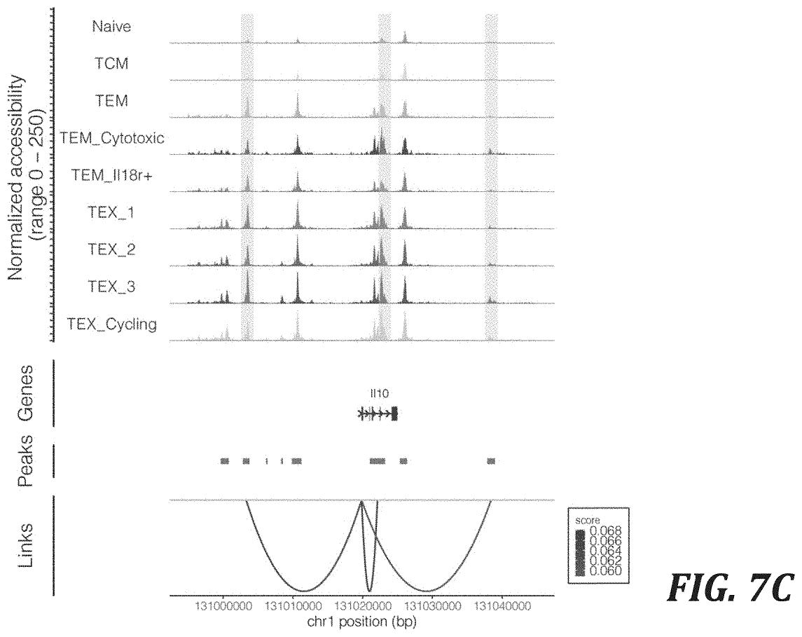

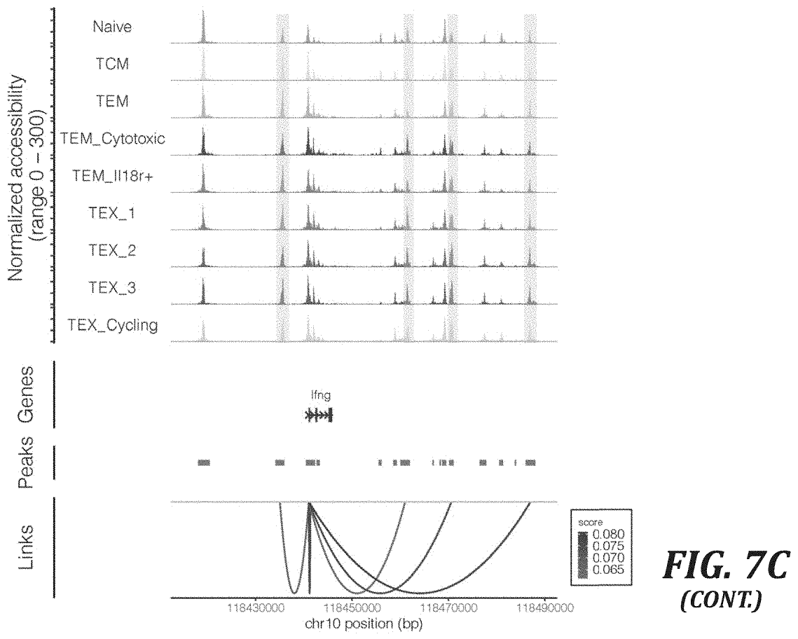

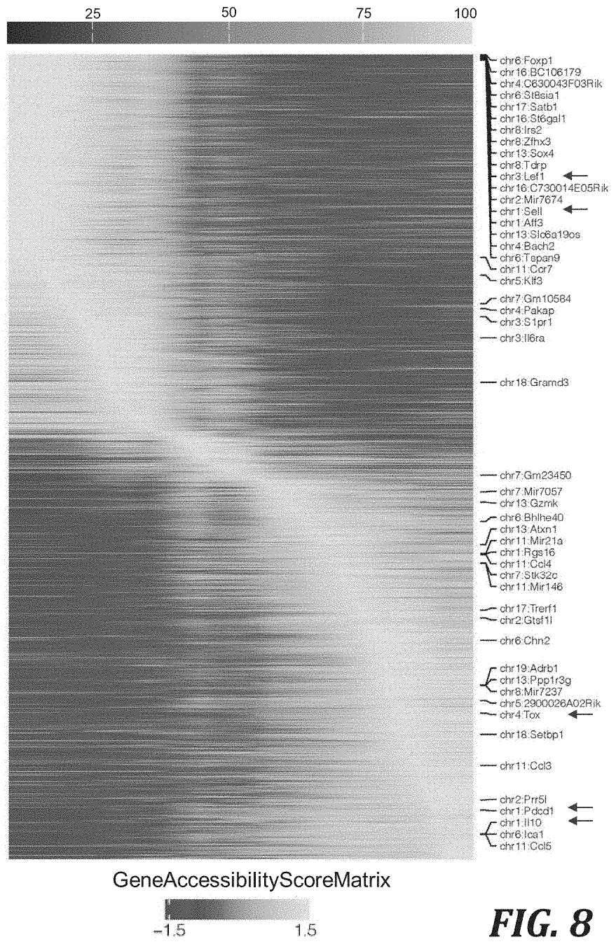

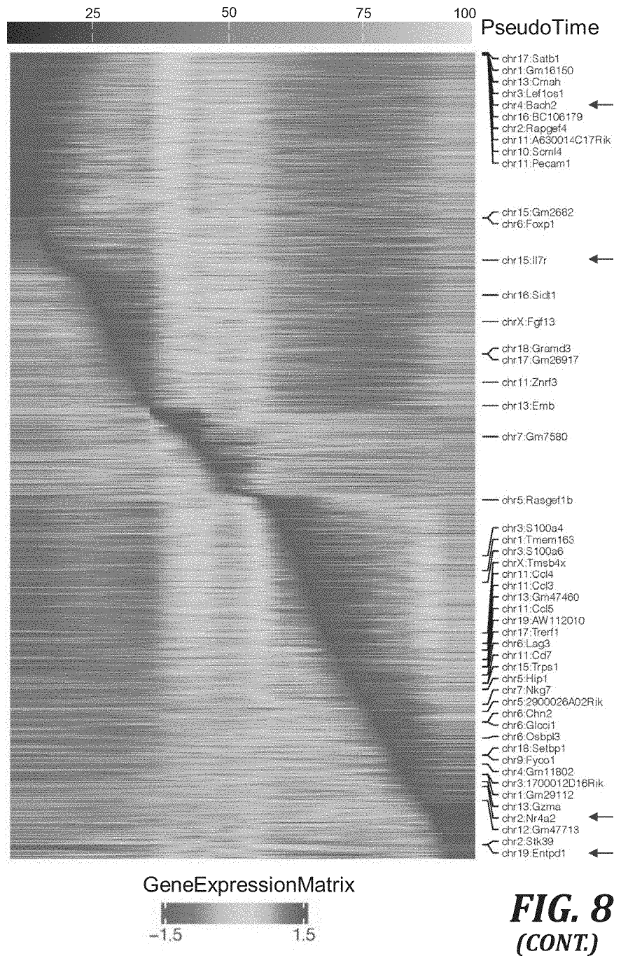

To determine whether TOX⁺T_{EFF} cells display epigenetic hallmarks of T-cell exhaustion, concurrent RNA and ATAC single cell sequencing (multiome) was performed on CD8 T-cells from the BM of MM-free, MM-controlled and MM-relapsed mice (FIG. 7A). CD8 T-cell clusters that were shared between all three cohorts as well as Tox⁺T_{EFF} clusters that were unique to mice with relapsed disease were observed (FIG. 2A). Using software capable of correlating measures of ATAC accessibility with expression of nearby genes in single cells, it was found that the Tox⁺T_{EFF}_1, Tox⁺T_{EFF}_2, and cell_cycling clusters had greater accessibility in Il10 and IFNγ linked peaks with an associated increase in gene expression (FIGS. 7B and 7C). This multiomic approach revealed that previously identified peaks that showed reduced accessibility across the IFNγ locus in TOX⁺ cells did not exhibit high levels of correlation with IFNγ gene expression in the datasets. A pseudotime trajectory from T_{N} to Tox⁺ cells was then created to identify transcriptomic and epigenetic differences across these transitioning cell states (FIG. 2B, FIG. 8). Naïve and stem-like genes were highly expressed and accessible at early points in the trajectory (Bach2, Lef1). Thereafter, a loss of stemness and gain of terminal/exhaustion signatures (Tox, Nr4a2, Pdcd1) was observed (FIG. 8). Surprisingly, in this trajectory, the highest levels of cytotoxic molecule (Prf1 and Gzmb) and IFNγ expression and accessibility were found in the Tox⁺T_{EFF}_2 cluster (FIG. 2C). Interestingly, maintained accessibility in, but not active transcription of these genes, was observed in putatively non-tumor specific Tox⁺ cells that accumulate at the end of the pseudotime trajectory, which were also found in MM-free mice (FIG. 2C). This cluster likely represents TCR-independent TOX expression that has been described in the context of inflammatory signals.

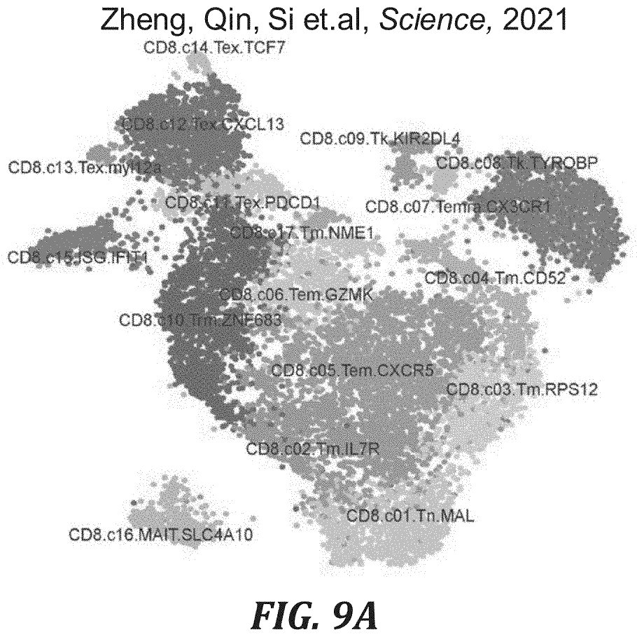

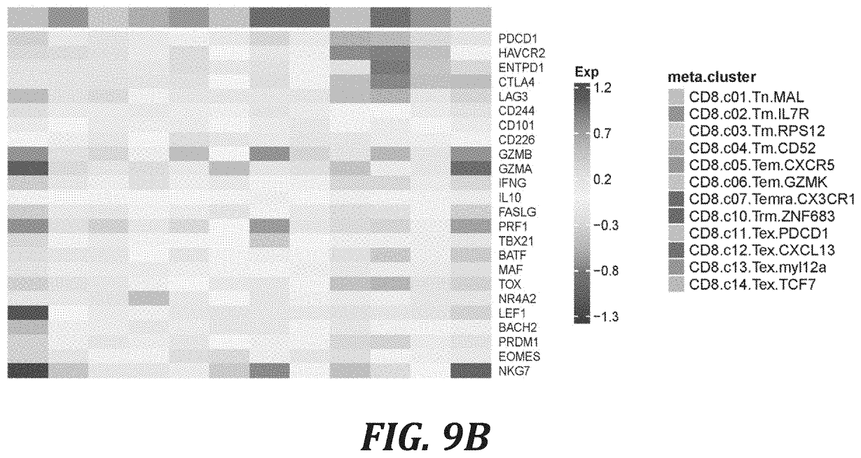

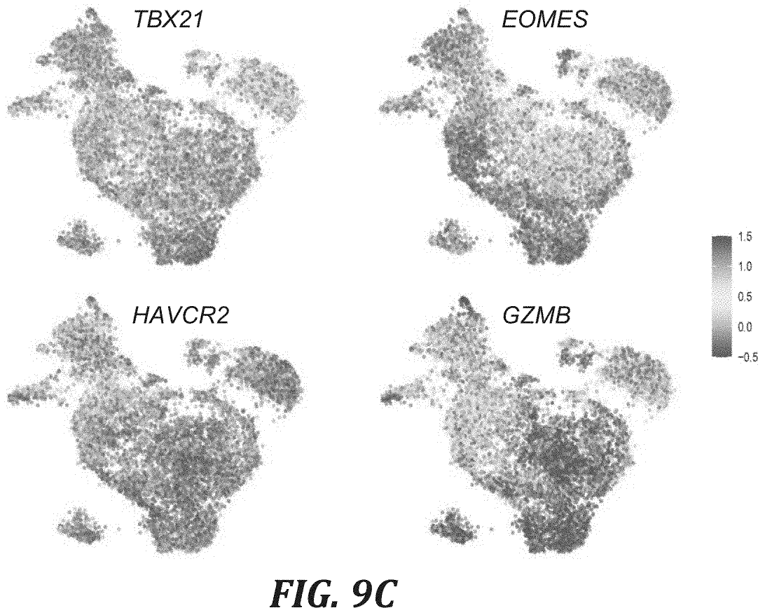

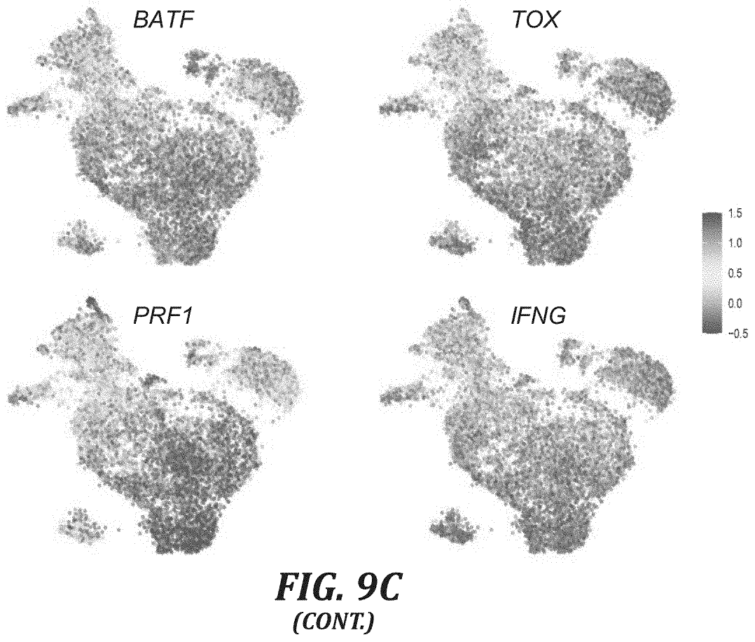

Transcription factor expression was then correlated with its motif enrichment in accessible DNA (buenrostro) to infer gene regulatory networks associated with TOX⁺T_{EFF} cells. With this approach, it was found that both positive and negative regulators of gene expression were present across the whole dataset. As expected, previously described regulators of T-cell exhaustion and stemness pathways had high regulation scores: Nr4a2, Eomes, Tcf7 (FIG. 2D). Interestingly, this analysis also highlighted Batf as a highly utilized transcription factor. Target genes of Batf and Eomes included those highly expressed in TOX⁺T_{EFF} cells, such as Pdcd1, Havcr, Prf1 and IFNγ (FIG. 2E). Furthermore, localized Batf and Eomes expression were observed in Tox-expressing cells, which had low Tbx21 expression, as well as co-expression of IFNγ and Il10 (FIG. 2F, FIG. 7B). Lastly, Tox-expressing cells with the highest level of IFNγ expression exhibited increased accessibility at the Batf motif (FIG. 2G, FIG. 7B). Together, these data suggest that TOX⁺ T-cells in the data, which exhibit an epigenetic profile consistent with an exhausted phenotype, also express high levels of Batf, a transcription factor characteristically associated with effector function. Importantly, the T_{EX} gene signature described in the mouse model (Tox, Havcr2, Pdcd1, Gzmb, IFNγ, Prf1) is also associated with BATF expression in tumor-infiltrating CD8 T-cells from a publicly available pan-cancer human dataset (FIGS. 9A, 9B, and 9C).

### A Decoy Resistant IL-18 Expands IFNγ-Secreting TOX⁺T_{EFF} Cells and Promotes Myeloma Control

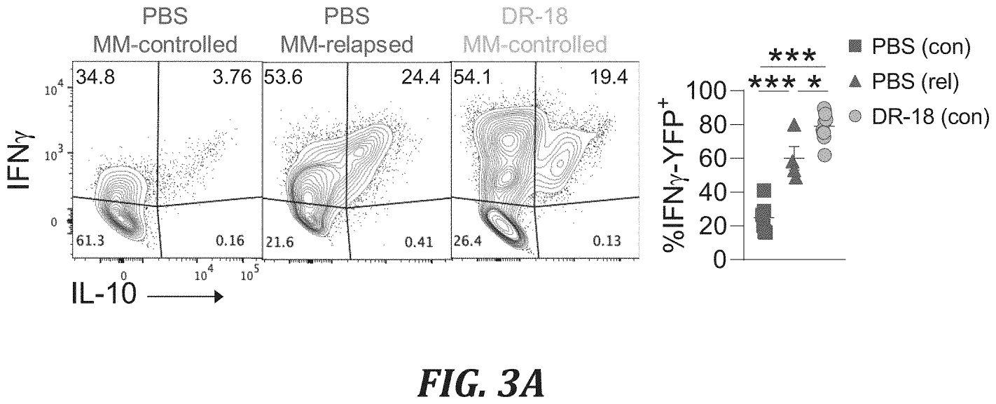

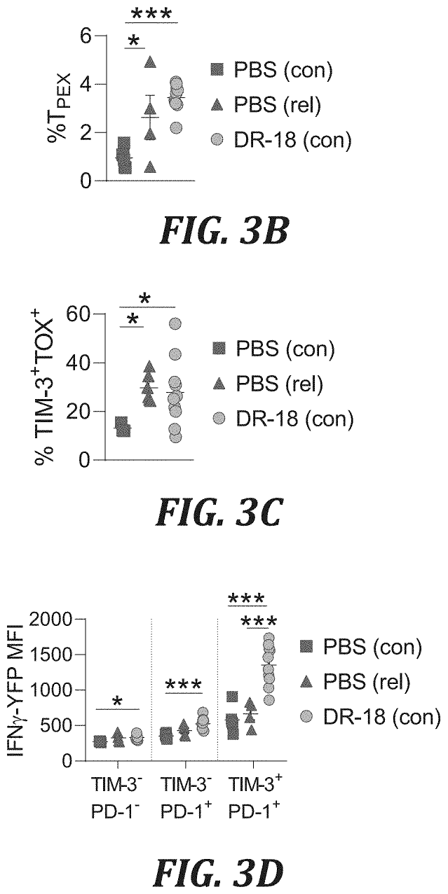

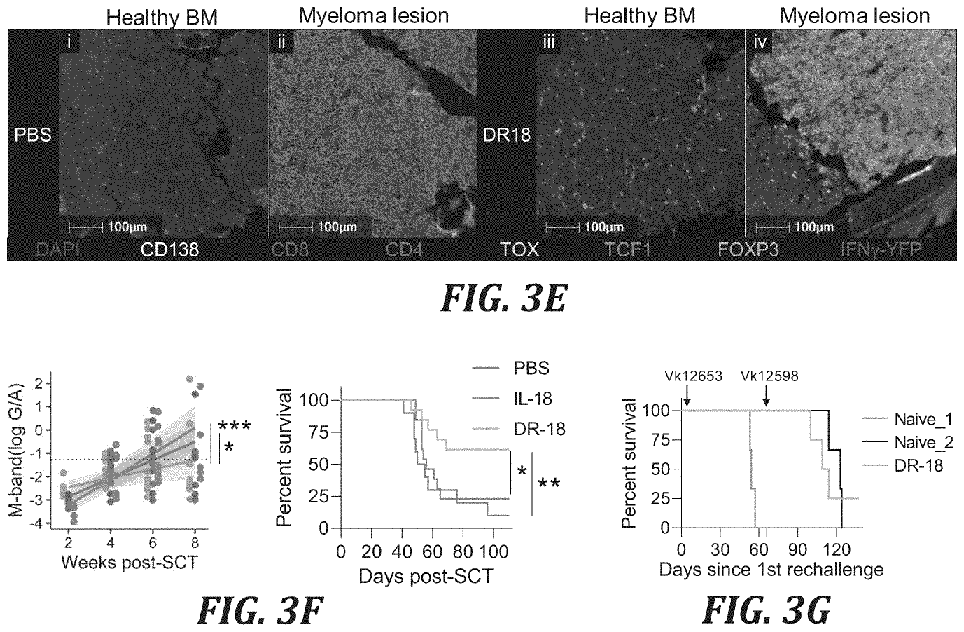

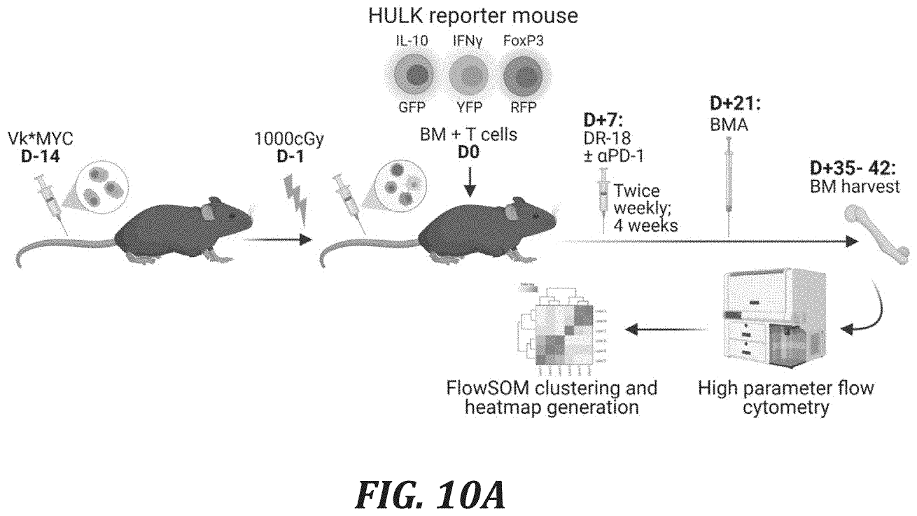

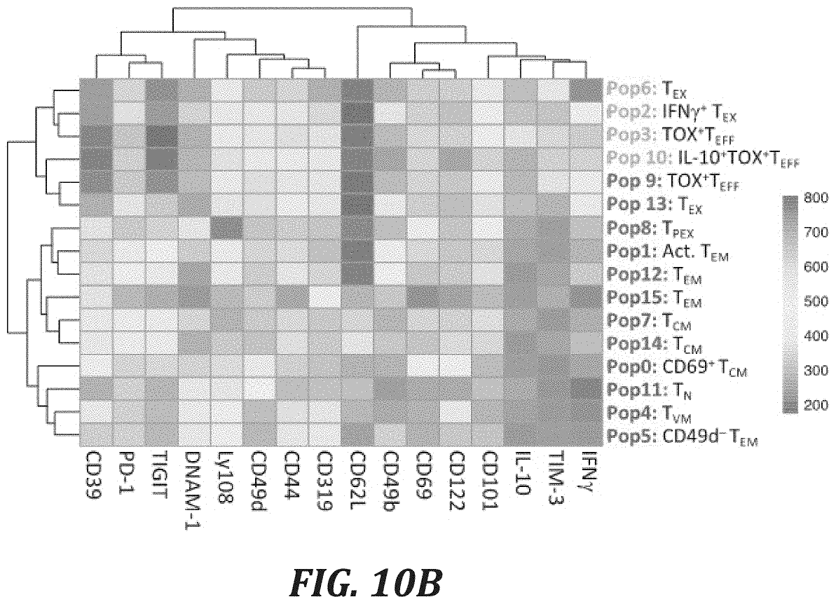

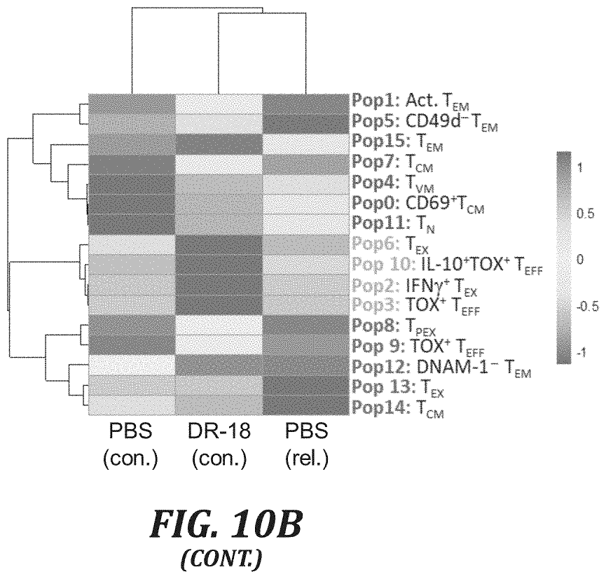

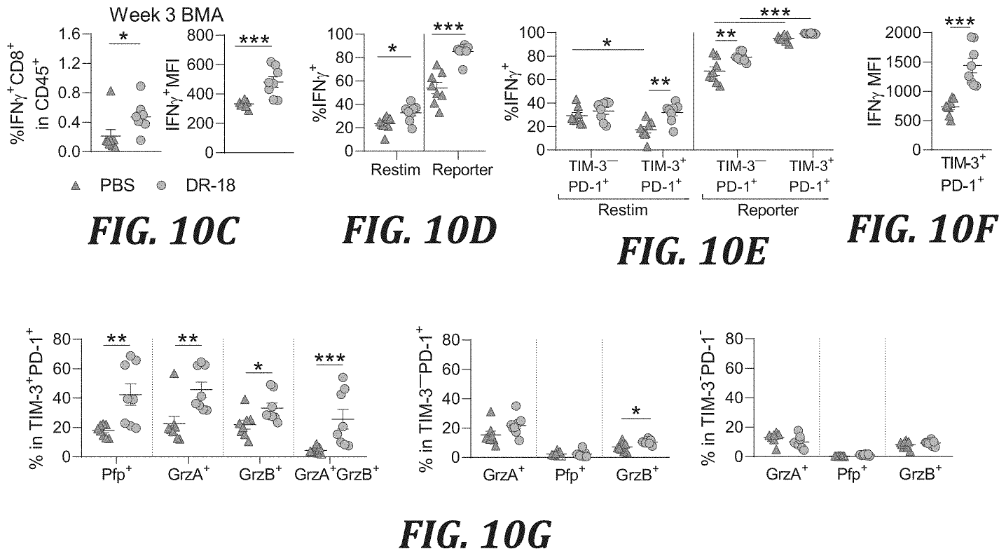

It was next determined whether TOX⁺T_{EFF} cells could mediate anti-tumor activity. Vk*MYC myeloma cells do not grow in culture and there are no described neoantigens to isolate known myeloma-specific T-cells. To address these limitations, it was sought to expand TOX⁺T_{EFF} cells in vivo with cytokines known to promote IFNγ. Expression of the IL-18 receptor in the dataset was observed (FIG. 5B) and therefore the agonist immunotherapy, decoy-resistant IL-18 (DR-18), was utilized. DR-18 has anti-tumor activity in solid tumor models and expands TIM-3⁺CD8 T-cells, although it does not upregulate TOX expression with the short-term treatment (8 days) utilized in those settings. In the model, mice were given IFNγ-reporter T-cells and were treated with DR-18 or PBS for 5 weeks (FIG. 10A). All DR-18 treated mice controlled the myeloma at 6 weeks post-transplant and this was associated with increased frequency of IFNγ⁺CD8 T-cells compared to PBS-treated mice (FIG. 3A). DR-18 treatment also expanded precursor exhausted (T_{PEX}) and TOX⁺T_{EFF} cells relative to PBS-treated mice with controlled myeloma (FIGS. 3B and 3C, FIG. 10B). PBS-treated mice with relapsed myeloma had a similar frequency of TOX⁺T_{EFF} cells to DR-18 treated mice but they produced significantly less IFNγ (FIG. 3D). These data suggest that increasing the frequency of TOX⁺T_{EFF} cells prior to states of high tumor burden may compensate for the lack of self-renewability of this population.

To address this hypothesis, CD8 T-cell function in the BM of DR-18 treated mice was determined at timepoints preceding high tumor burden by non-terminal BM aspirates. DR-18 increased the frequency of IFNγ⁺CD8 T-cells and IFNγ MFI as early as 3 weeks post-transplant, which remained elevated at 5 weeks post-transplant in the same mice (FIGS. 10C, 10D, 10E, 10F). DR-18-driven enhancement of IFNγ was detectable by PMA/ionomycin stimulation, however IFNγ expression in TIM-3⁺ cells was particularly underestimated by this approach (FIG. 10E). DR-18 also increased expression of cytotoxic molecules in TIM-3⁺ cells (FIG. 10G). Spatial analysis of BM from DR-18 and PBS treated mice was then performed to measure tumor lesion infiltration. Mutiplex-immunohistochemistry (M-IHC) revealed infiltration of IFNγ⁺TOX⁺ T-cells in myeloma lesions that was enhanced by DR-18 treatment as well as accumulation of T_{PEX} at the tumor margin (FIG. 3E). Furthermore, immune cell infiltration, including TOX⁺ T-cells, was increased in areas of healthy BM in response to DR-18 (FIG. 3E). Importantly, DR-18 significantly decreased myeloma burden and improved overall survival while native IL-18 had no anti-myeloma effect (FIG. 3F). DR-18-mediated tumor immunity was highly specific for the myeloma clone present at the time of treatment and cured mice were protected from rechallenge with the original myeloma, but challenge with an unrelated myeloma clone resulted in uncontrolled tumor growth (FIG. 3G). The data suggest that the dramatically increased ratio of highly functional TOX⁺ cells to myeloma likely underpins the anti-tumor efficacy of DR-18. Furthermore, TOX⁺T_{EFF} cells in DR-18 responsive mice were characterized by increased production of granzymes, perforin and IFNγ.

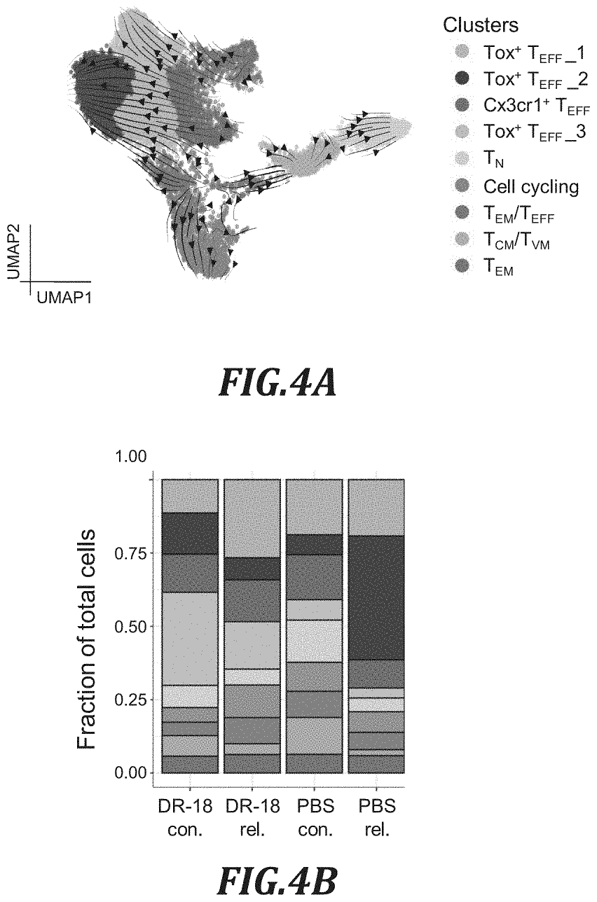

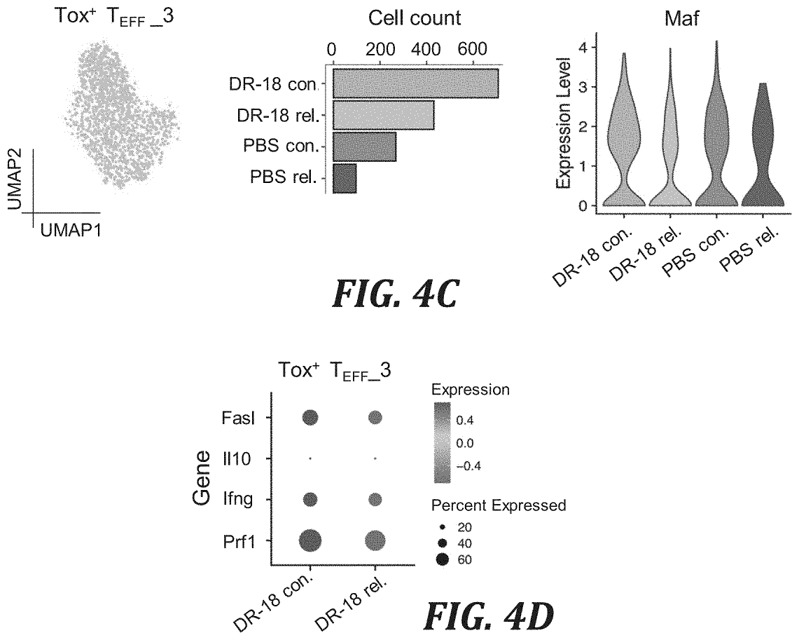

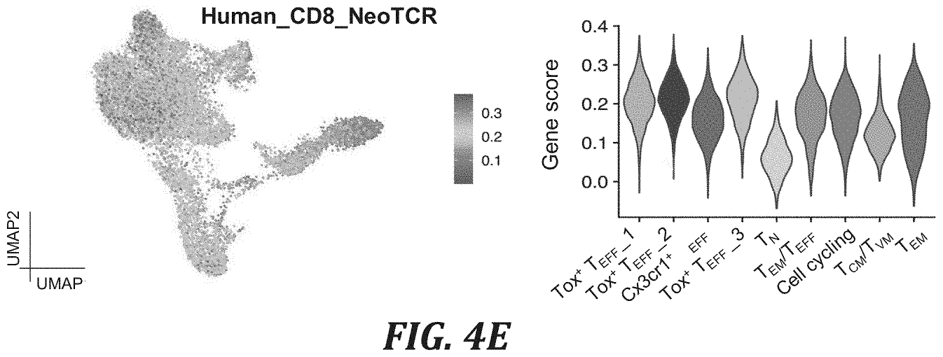

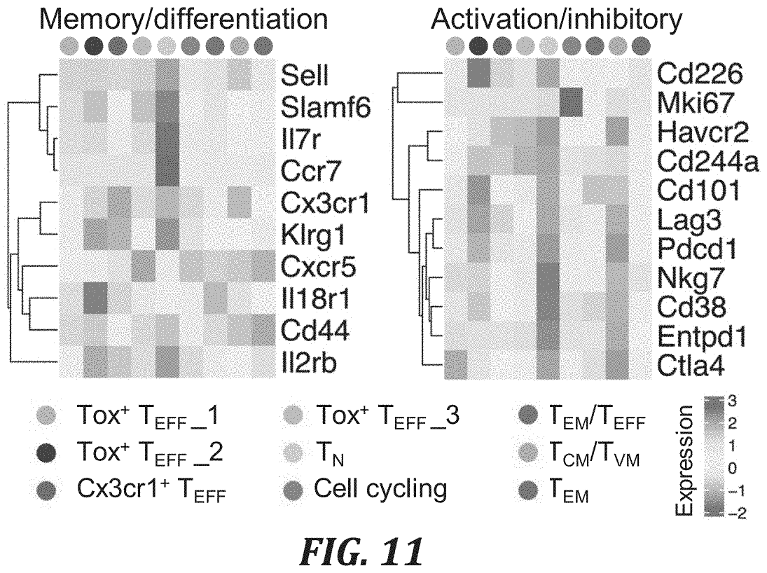

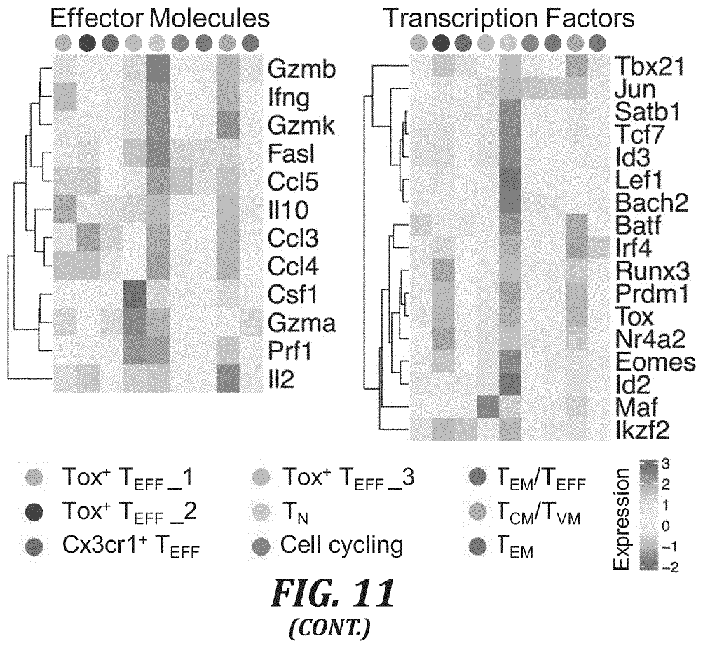

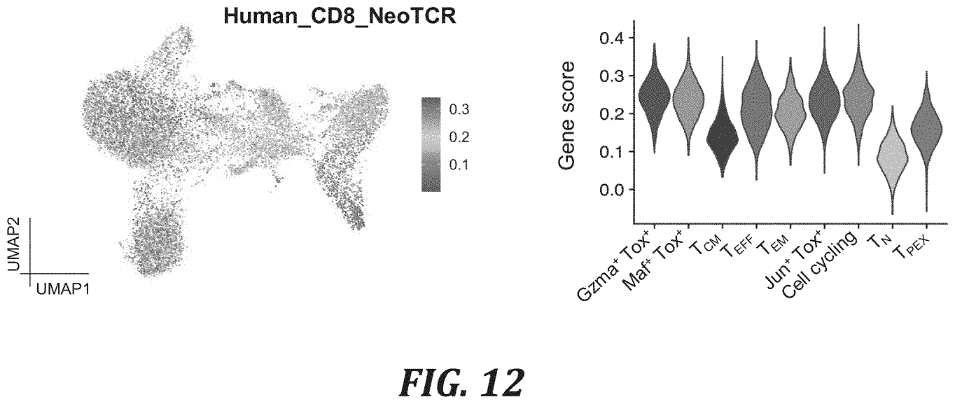

To understand why some mice do not respond to DR-18, single cell RNA sequencing on CD8 T-cells from DR-18 treated mice with controlled myeloma (DR-18_con) or mice with an M-band above the relapse threshold (DR-18 rel) compared to respective PBS controls was performed. Nine (9) clusters were identified, including three Tox⁺T_{EFF} clusters (FIG. 4A and FIG. 11). Interestingly, the Tox⁺T_{EFF}_3 cluster demonstrated divergence from the other TOX⁺T_{EFF} clusters by RNA velocity analysis (FIG. 4A). Despite similar overall expansion of TOX⁺T_{EFF} cells, DR-18 resistance was associated with a relative loss of Tox⁺T_{EFF}_3, a cluster with high expression of Maf and cytotoxic genes including perforin (FIGS. 4B, 4C, 4D, FIG. 11). Rather, DR-18 rel mice were enriched for the Tox⁺T_{EFF}_1 cluster with high IFNγ but low Prf1 gene expression. These data provide further evidence for the diverse functional capacity of TOX⁺ T-cell subsets and highlight the importance of both IFNγ and cytotoxic molecules in mediating the most potent anti-tumor responses. Lastly, the expression of genes that have recently been associated with tumor-specific T-cells in human cancers was evaluated and robust expression of this gene set in TOX⁺T_{EFF} clusters both with DR-18 treatment and in the initial dataset from myeloma-relapsed mice was observed (FIG. 4E, FIG. 12). In conclusion, subsets of TOX⁺ T-cells identified in the data display hallmarks of T-cell exhaustion and tumor-specificity but are highly functional and are further expanded during DR-18 treatment.

### Discussion

In this example, highly functional TIM-3⁺TOX⁺ T-cells in the bone marrow TME that lack self-renewability and display hallmarks of T-cell exhaustion are described. Expansion of these cells with DR-18 promoted tumor control and DR-18 resistance was associated with loss of a Maf-expressing TOX⁺T_{EFF} subset that had high perforin expression. Spatial imaging of the BM TME demonstrated that loss of T-cell-mediated control in the model was underpinned by a reduced ratio of functional T-cells to tumor cells in the BM rather than dysfunction of tumor-infiltrating T-cells per se. It is important to highlight that while these cells can mediate anti-tumor activity they do not have features of self-renewability and are therefore unlikely to generate sustainable anti-tumor immunity. The effectiveness of DR-18 in generating long-term protection against tumor relapse was associated with the concurrent expansion of TOX⁺T_{EFF} and T_{PEX} cells as well as the accelerated differentiation of highly functional TOX⁺T_{EFF}. Importantly, this process temporally precedes the accumulation of tumor cells in the BM.

The dogma in the T-cell exhaustion field, that cytokine production is severely limited in T-cells expressing TOX and multiple inhibitory receptors, is built on data generated through ex vivo re-stimulation. Previously published data also demonstrate reduced IFNγ production after PMA/ionomycin restimulation in MM-relapsed mice but robust IFNγ production in MM-free mice. These results directly contradict the current example's findings which utilized an in vivo IFNγ reporter system. The discrepancy between high versus low cytokine secretion in vivo versus ex vivo following restimulation with PMA/ionomycin likely reflects a re-stimulation artefact, and not post-translational modification of IFNγ RNA, to which it has been previously ascribed. This is likely driven by high expression of negative feedback regulators of the IFNγ pathway in effector T-cells or defects in PMA/ionomycin response elements, that are bypassed in vivo where physiological IFNγ responses are primarily determined by IL-12/IL-18 responses. Furthermore, the co-expression of TOX and BATF in both humans and mice, that is associated with the expression of genes known to confer anti-tumor immunity in these TOX⁺T_{EFF} subsets, is demonstrated herein. Importantly, BATF has been described as a driver of both effector function and exhaustion in different biological contexts. This data suggest that these phenotypes are not mutually exclusive and that Batf correlates with enhanced cytokine and granzyme production in exhausted-like TOX⁺T_{EFF} T-cells.

In conclusion, TIM-3⁺TOX⁺CD8 T-cells are identified and characterized herein. These cells have hallmarks of exhaustion and are highly functional in the TME and are expanded using methods and compositions of the present disclosure to enhance anti-tumor activity. Thus, the function, or lack thereof, of T-cells in the TME cannot be inferred by measuring expression of inhibitory receptors or even exhaustion-associated transcription factors. These data have implications for all studies investigating CD8 T-cell phenotypes in tumor settings, particularly as this phenotype was observed in human TILs from diverse cancers. Finally, this data emphasize the importance of T_{PEX} in generating sustained responses to immunotherapies, rather than the reversal of dysfunction within a terminally “exhausted” T-cell.

### Methods

### Mice

C57BL/6j mice were purchased from Jackson Laboratory (Bar Harbor, Me., USA). PTP×C57 (CD45.1/CD45.2) and HULK (IFNγ-YFP×IL-10-GFP×FoxP3-RFP) reporter mice were bred in house at Fred Hutchinson Cancer Research Center (Seattle, Wash., USA). Mice were housed in sterile microisolator cages, fed normal chow, and given autoclaved pH 2.5 water. All animal procedures were performed in accordance with protocols approved by the institutional animal ethics committee.

### Antibodies

The following antibodies for flow cytometry were purchased from: eBiosciences; CD101 (Moushil01), c-MAF (sym0F1), TIM3 (CD366; RMT3-23), FOXP3 (FJK-16s), TOX (TXRX10) and from Biolegend; Perforin (S16009A), CD319 (SLAMF7; 4G2), PD-1 (CD279; RMPI-30, 29F.1A12), CD38 (T10), CD39 (Duha59), CD90.2 (53-2.1), CD49d (R1-2), Granzyme B (QA16A02), CD69 (H1.2F3), CD226 (DNAM-1; TX42.1), CD3 (145-2C11) and from BD Bioscience; CD122 (TM-(31), Granzyme B (GB11), CD44 (IM7), TIGIT (1G9), CD62L (MEL-14), CD45 (30-F11), CD45.1 (A20), CD4 (GK1.5), LY108 (13G3), PD-1 (CD279; J43), CD8 (53-6.7).

The following antibodies were used for immunohistochemistry: GFP (rabbit polyclonal; Invitrogen; 1:1000; position 1) with Powervision Rabbit-HRP Opal 570 secondary, CD8a (Rabbit D4W2Z; Cell Signaling Technology; 1:3000; position 2) with Powervision Rabbit-HRP Opal 620 secondary, CD19 (Rat 60MP31; eBioscience; 1:800; position 3) with ImmPress Rat-HRP Opal 650 secondary, TOX (Rat TXRX10; eBioscience; 1:100; position 4) with ImmPress Rat-HRP Opal 540 secondary, CD138 (Rat 281-2; Biolegend; 1:800; position 5) with ImmPress Rat-HRP Opal 520 secondary, CD4 (Rat 4SM95; eBioscience; 1:500; position 6) ImmPress Rat-HRP Opal 480 secondary, FoxP3 (Rabbit 1054C; R&D; 1:600; position 7) with Powervision Rabbit-HRP Opal 690 secondary, TCF1/TCF7 (Rabbit C63D9; 1:200; position 8) with Powervision Rabbit-HRP Opal 780 secondary.

### Myeloma Model and Stem Cell Transplantation

Mice were injected intravenously with Vk12653 or Vk12598 (1×10⁶ cells; MM-bearing mice), which originated from Vk*MYC transgenic mice. For transplantation experiments, recipients were injected with Vk*MYC two weeks or were naïve (MM-free) prior to lethal irradiation split across two doses 3 hours apart (1000 cGy, 137Cs source). Recipients were transplanted the following day with 10×10⁶ BM and 5×10⁶ T-cells via tail vein injection as described previously. PTP×C57 recipients were utilized in all experiments with HULK reporter donors to allow for the exclusion of reporter negative recipient T-cells from downstream analysis. Serum samples were collected every two weeks from MM-bearing recipients and M-band was quantified using serum protein electrophoresis (HYDRASYS 2 Scan) as previously described. Mice were treated from D+7 for 4-5 weeks with 8 μg DR-18 (as described) s.c. twice a week. Recipients were monitored daily and sacrificed when clinical scores reached 36 or hind limb paralysis was observed. For the rechallenge experiment, mice with long-term myeloma control after DR-18 treatment were injected with the myeloma clone (Vk12563) that they were originally transplanted with. A cohort of naïve mice were injected at the same time as controls for unconstrained tumor growth. The long-term survivors who were protected in the first rechallenge were subsequently injected with a different myeloma clone (Vk12598) at the same time as a new cohort of naïve mice.

### Cell Preparation for Flow Cytometry

Mice were sacrificed at time points indicated in Figure Legends and BM was flushed with media (RPMI+1% FCS) to harvest T-cells. For bone marrow aspirates, mice were anesthetized and treated with a local analgesic (0.5% lidocaine) followed by injection of 30 μL of PBS into the femur to allow up to 10 μL of marrow to be aspirated. For FACS analysis, whole BM was incubated with Fc-block prior to staining with fluorescently tagged antibodies on ice for 30 minutes. For intracellular staining, cells were permeabilized after surface staining (Foxp3 Staining Buffer Kit; eBioscience) prior to incubation with intracellular antibodies for 60 minutes at room temperature. To measure restimulated IFNγ production, cells were stimulated with PMA (500 ng/mL) and ionomycin (50 ng/mL) (Sigma-Aldrich) with Brefeldin A (BioLegend) for 4 hours at 37° C. All samples were acquired on a BD FACSymphony A3 (BD Biosciences) and analyzed using FlowJo software (v10). FlowSOM analysis was performed with 4000 CD8 T-cells per mouse concatenated after downsampling

### Multiplex Immunohistochemistry

Formalin-fixed paraffin-embedded tissues were sectioned at 4 microns onto positively-charged slides and baked for 1 hour at 60° C. The slides were then dewaxed and stained on a Leica BOND Rx stainer (Leica, Buffalo Grove, Ill.) using Leica Bond reagents for dewaxing (Dewax Solution), antigen retrieval/antibody stripping (Epitope Retrieval Solution 2) and rinsing after each step (Bond Wash Solution). Antigen retrieval and antibody stripping steps were performed at 100° C. with all other steps at ambient temperature.

Endogenous peroxidase was blocked with 3% H₂O₂ for 5 minutes followed by protein blocking with 10% normal mouse immune serum diluted in TCT buffer (0.05M Tris, 0.15M NaCl, 0.25% Casein, 0.1% Tween 20, 0.05% ProClin300 pH 7.6) for 10 minutes. The first primary antibody (position 1) was applied for 60 minutes followed by the secondary antibody application for 20 minutes and the application of the tertiary TSA-amplification reagent (PerkinElmer OPAL fluor) for 20 minutes. A high stringency wash was performed after the secondary and tertiary applications using high-salt TBST solution (0.05M Tris, 0.3M NaCl, and 0.1% Tween-20, pH 7.2-7.6). Species specific Polymer HRP was used for all secondary applications, either Leica's PowerVision Poly-HRP anti-Rabbit Detection or ImmPress Goat anti-Rat IgG Polymer Detection Kit (Vector Labs, Burlingame, Calif.). The primary and secondary antibodies were stripped with retrieval solution for 20 minutes before repeating the process with the second primary antibody (position 2) starting with a new application of 3% H2O2. The process was repeated until seven positions were completed. For the eighth position, following the secondary antibody application, Opal TSA-DIG was applied for 20 minutes, followed by the 20-minute stripping step in retrieval solution and application of Opal 780 fluor for 60 minutes with high stringency washes performed after the secondary, TSA DIG, and Opal 780 fluor applications. The stripping step was not performed after the final position. Slides were removed from the stainer and stained with DAPI for 5 minutes, rinsed, and coverslipped with Prolong Gold Antifade reagent (Invitrogen/Life Technologies, Grand Island, N.Y.).

Slides were cured at room temperature, then whole slide images were acquired on the Vectra Polaris Quantitative Pathology Imaging System (Akoya Biosciences, Marlborough, Mass.). The entire tissue was selected for imaging using Phenochart and multispectral image tiles were acquired using the Polaris. Images were spectrally unmixed using Phenoptics inForm software and exported as multi-image TIF files.

Images were analyzed with HALO image analysis software (Indica Labs, Cooales, N. Mex.) using the Highplex FL module, v3.2.1. Cellular analysis of the images was performed by first identifying cells based on nuclear recognition (DAPI stain), then measuring fluorescence intensity of the estimated cytoplasmic areas of each cell. A mean intensity threshold above background was used to determine positivity for each fluorochrome within the cytoplasm, thereby, defining cells as either positive or negative for each marker. The positive cell data was then used to define colocalized populations.

### Single-Cell RNA Sequencing CD8⁺ T-Cells from MM-Relapsed Mice and DR18 Treated Mice

For all sequencing experiments, BM was harvested from femurs and T-cell populations were FACS sorted prior to sample preparation according to 10× Genomics protocols. In some experiments, individual mice or purified T-cell populations were stained with BioLegend Hashtag TotalSeqB reagents, and cell surface markers (including PD-1, TIM-3, CD4 and CD8), according to manufacturer's protocol to allow pooling of samples for capture. T-cells (gene expression) or nuclei (multiome, also herein referred to as cells) were captured and libraries were generated according to manufacturer's specifications. Libraries were sequenced using an Illumina NovaSeq 6000 or NextSeq 2000 targeting a depth of 25,000 reads per cell per library. Cellranger was used to process reads and output was imported into a Monocle3 cell data set (CDS). Counts of antibody hashes generated using cellranger were used to remove putative multiplets. Scrublet was also used to predict and remove doublets. The resulting dataset was further filtered to remove cells with more than 20000 or less than 2000 UMI per cell, and cells with a percent of mitochondrial reads >5%. An initial UMAP embedding was generated from UMI-corrected counts of the 2000 most variant genes first subject to PCA to using 25 components. Satellite clusters containing CD4 CD8 double positive cells were then removed (CD8+ MM-relapsed dataset only). Iterative latent semantic indexing and clustering was used to create the final embedding as previously described with 3 total iterations using a clustering resolution of 10⁻³ (all iterations), 20 dimensions of LSI (all iterations), and 4000 input features for the first iteration and 3000 for the last two. Clusters were identified using a final resolution of 10⁻² (CD8+ MM-relapsed mice) and 5×10⁻³ (DR18 and PBS control). For the DR18/PBS dataset, a Cd8 T-cell subset was made by finding clusters and sub-setting a partition with distinct Cd8 expression. Cells were re-embedded with the same parameters as above.

To estimate and visualize RNA velocity, matrices of spliced and unspliced counts were generated using the velocyto command line tool with default options using the ‘run10×’ function on cellranger output. Scvelo was used to render RNA velocity using default settings and visualized on the UMAP embedding generated from iterative latent semantic indexing. Publicly available data was downloaded and made into CDS objects and projected as previously described. Gene set scores were calculated as previously described.

### Single-Cell RNA/ATAC Analysis

Reads were demultiplexed and processed using cellranger. Peaks were called from each sample's fragments.tsv file (cellranger output) using MACS2 using the parameters ‘--nomodel--extsize 200--shift-100’. Quantification of reads in MACS2 peaks were calculated and integrated with RNA counts using Signac. Cells meeting the following criteria (calculated using Signac) were retained for downstream analysis: percent mitochondrial RNA reads <15%; 3<log₁₀(ATAC counts)<5; 3<log₁₀(RNA/UMI counts)<4.5; Fraction of reads in peaks>40%. RNA/UMI counts were subject to a variance-stabilized normalization procedure using Seurat's ‘glmGamPoi’ function prior to dimensionality reduction using PCA. ATAC data after TFIDF/SVD dimensionality reduction and batch correction with Harmony were integrated with reduced-dimensionality RNA data (PCA matrix) using the WNN function using default parameters. Clusters were identified using the standard Seurat workflow. CD8 negative clusters were removed. Peak to gene linkages were calculated with the ‘LinkPeaks’ function and motif accessibility was calculated using the RunChromVAR function. Gene activity scores and pseudotime trajectory analysis were performed with ArchR.

### Human single-Cell RNA Sequencing Data

The human data used to show BATF expression in T_{EX} is from a publicly available source and figures were generated using the CD8 dataset in the online tool available at http://cancer-pku.cn:3838/PanC_T/. The human neoantigen TCR dataset was from Lowery et al. and was incorporated into the data as described above.

### Statistical Analysis

Survival curves were plotted using Kaplan-Meier estimates and compared by Log-rank (Mantel-Cox) test. M-bands were modeled as described previously and the M-band relapse threshold (G/A above 0.282) has been reported. Comparisons between two groups were performed with Mann-Whitney U test or t-test and those between three or more groups were performed with Kruskal-Wallis and Dunn's multiple comparisons test or one-way ANOVA and Tukey's multiple comparisons test for nonparametric data and normally distributed data respectively. All data presented as mean±SEM and p<0.05 was considered significant.

### EXEMPLARY EMBODIMENTS

Embodiment 1. A method for treating a malignancy in a subject, the method comprising contacting a composition to enrich for T_{PEX} cells and/or TOX⁺T_{EFF} cells to a plurality of T cells to enhance anti-tumor activity of the plurality of T cells to treat the malignancy.

Embodiment 2. The method of any other embodiment, wherein the composition comprises an agonist immunotherapy or a decoy-resistant IL-18 (DR-18) immunotherapy.

Embodiment 3. The method of any other embodiment, wherein the contacting the composition expands IFNγ⁺TOX⁺T_{EFF} cells and promotes tumor-specific immunity.

Embodiment 4. The method of any other embodiment, wherein the contacting the composition expands Maf-expressing TOX⁺T_{EFF} cells with a tumor-specific gene signature.

Embodiment 5. The method of any other embodiment, wherein the TOX⁺T_{EFF} cells express Basic leucine zipper transcription factor, ATF-like (BATF).

Embodiment 6. The method of any other embodiment, wherein the composition is administered to the subject in vivo and enrichment for T_{PEX} cells and/or TOX⁺T_{EFF} cells in the plurality of T cells occurs in vivo.

Embodiment 7. The method of any other embodiment, wherein the administering the composition to the subject increases survival of the subject relative to not administering the composition.

Embodiment 8. The method of any other embodiment, wherein the administering the composition occurs prior to a state of high tumor burden of the malignancy.

Embodiment 9. The method of any other embodiment, further comprising administering a restimulation composition to restimulate the plurality of T cells.

Embodiment 10. The method of any other embodiment, wherein the restimulation composition comprises phorbol 12-myristate 13-acetate (PMA) and ionomycin.

Embodiment 11. A method for determining whether enhancement of an anti-tumor activity of a plurality of T cells associated with a tumor microenvironment (TME) of a malignancy occurs in a subject, the method comprising: administering a composition to the subject to enrich for T_{PEX} cells and/or TOX⁺T_{EFF} cells in the plurality of T cells; and determining, with an assay, whether the anti-tumor activity is enhanced as a result of administering the composition.

Embodiment 12. The method of any other embodiment, wherein the TOX⁺T_{EFF} cells express Basic leucine zipper transcription factor, ATF-like (BATF).

Embodiment 13. The method of any other embodiment, wherein the composition comprises an agonist immunotherapy or a decoy-resistant IL-18 (DR-18) immunotherapy.

Embodiment 14. The method of any other embodiment, wherein the administering the composition expands IFNγ⁺TOX⁺T_{EFF} cells and promotes tumor-specific immunity, and expands Maf-expressing TOX⁺T_{EFF} cells with a tumor-specific gene signature.

Embodiment 15. The method of any other embodiment, wherein the composition is administered to the subject in vivo and enrichment for T_{PEX} cells and/or TOX⁺T_{EFF} cells in the plurality of T cells occurs in vivo.

Embodiment 16. The method of any other embodiment, wherein the administering the composition occurs prior to a state of high tumor burden of the malignancy.

Embodiment 17. The method of any other embodiment, wherein the subject is a cytokine reporter animal model.

Embodiment 18. The method of any other embodiment, wherein the assay measures at least one reporter protein of the cytokine reporter animal model for determining whether enhancement of the anti-tumor activity occurs as a result of administering the composition to the subject.

Embodiment 19. The method of any other embodiment, further comprising administering a restimulation composition to restimulate the plurality of T cells.

Embodiment 20. The method of any other embodiment, wherein the restimulation composition comprises phorbol 12-myristate 13-acetate (PMA) and ionomycin.

While illustrative embodiments have been illustrated and described, it will be appreciated that various changes can be made therein without departing from the spirit and scope of the disclosure.

The embodiments of the disclosure in which an exclusive property or privilege is claimed are defined as follows:

1. A method for treating a malignancy in a subject, the method comprising contacting a composition to enrich for T_{PEX} cells and/or TOX⁺T_{EFF} cells to a plurality of T cells to enhance anti-tumor activity of the plurality of T cells to treat the malignancy.

2. The method of claim 1, wherein the composition comprises an agonist immunotherapy or a decoy-resistant IL-18 (DR-18) immunotherapy.

3. The method of claim 1, wherein the contacting the composition expands IFNγ⁺TOX⁺T_{EFF} cells and promotes tumor-specific immunity.

4. The method of claim 1, wherein the contacting the composition expands Maf-expressing TOX⁺T_{EFF} cells with a tumor-specific gene signature.

5. The method of claim 1, wherein the TOX⁺T_{EFF} cells express Basic leucine zipper transcription factor, ATF-like (BATF).

6. The method of claim 1, wherein the composition is administered to the subject in vivo and enrichment for T_{PEX} cells and/or TOX⁺T_{EFF} cells in the plurality of T cells occurs in vivo.

7. The method of claim 6, wherein the administering the composition to the subject increases survival of the subject relative to not administering the composition.

8. The method of claim 6, wherein the administering the composition occurs prior to a state of high tumor burden of the malignancy.

9. The method of claim 1, further comprising administering a restimulation composition to restimulate the plurality of T cells.

10. The method of claim 9, wherein the restimulation composition comprises phorbol 12-myristate 13-acetate (PMA) and ionomycin.

11. A method for determining whether enhancement of an anti-tumor activity of a plurality of T cells associated with a tumor microenvironment (TME) of a malignancy occurs in a subject, the method comprising:
administering a composition to the subject to enrich for T_{PEX} cells and/or TOX⁺T_{EFF} cells in the plurality of T cells; and
determining, with an assay, whether the anti-tumor activity is enhanced as a result of administering the composition.

12. The method of claim 11, wherein the TOX⁺T_{EFF} cells express Basic leucine zipper transcription factor, ATF-like (BATF).

13. The method of claim 11, wherein the composition comprises an agonist immunotherapy or a decoy-resistant IL-18 (DR-18) immunotherapy.

14. The method of claim 11, wherein the administering the composition expands IFNγ⁺TOX⁺T_{EFF} cells and promotes tumor-specific immunity, and expands Maf-expressing TOX⁺T_{EFF} cells with a tumor-specific gene signature.

15. The method of claim 11, wherein the composition is administered to the subject in vivo and enrichment for T_{PEX} cells and/or TOX⁺T_{EFF} cells in the plurality of T cells occurs in vivo.

16. The method of claim 11, wherein the administering the composition occurs prior to a state of high tumor burden of the malignancy.

17. The method of claim 11, wherein the subject is a cytokine reporter animal model.

18. The method of claim 17, wherein the assay measures at least one reporter protein of the cytokine reporter animal model for determining whether enhancement of the anti-tumor activity occurs as a result of administering the composition to the subject.

19. The method of claim 11, further comprising administering a restimulation composition to restimulate the plurality of T cells.

20. The method of claim 19, wherein the restimulation composition comprises phorbol 12-myristate 13-acetate (PMA) and ionomycin.
